SEQUENCE LISTING (258 sequences)

SEQ ID NO: 1 - DNA, 21 bp, synthetic construct
  source 1..21: mol_type = other DNA; note = Description of Artificial Sequence: Synthetic primer
catcagaccggcagattaacc

SEQ ID NO: 2 - DNA, 21 bp, synthetic construct
  source 1..21: mol_type = other DNA; note = Description of Artificial Sequence: Synthetic primer
gttggccttacagcggtagat

SEQ ID NO: 3 - DNA, 21 bp, synthetic construct
  source 1..21: mol_type = other DNA; note = Description of Artificial Sequence: Synthetic primer
gtggctgacctggtacaagag

SEQ ID NO: 4 - DNA, 22 bp, synthetic construct
  source 1..22: mol_type = other DNA; note = Description of Artificial Sequence: Synthetic primer
ggtagatgcgtctgagttccat

SEQ ID NO: 5 - DNA, 19 bp, synthetic construct
  source 1..19: mol_type = other DNA; note = Description of Artificial Sequence: Synthetic primer
gaggccaagccctggtatg

SEQ ID NO: 6 - DNA, 19 bp, synthetic construct
  source 1..19: mol_type = other DNA; note = Description of Artificial Sequence: Synthetic primer
cgggccgattgatctcagc

SEQ ID NO: 7 - DNA, 21 bp, synthetic construct
  source 1..21: mol_type = other DNA; note = Description of Artificial Sequence: Synthetic primer
acagttgcagccgtagtcttg

SEQ ID NO: 8 - DNA, 20 bp, synthetic construct
  source 1..20: mol_type = other DNA; note = Description of Artificial Sequence: Synthetic primer
ccaggtcgttgtgagcttct

SEQ ID NO: 9 - DNA, 21 bp, synthetic construct
  source 1..21: mol_type = other DNA; note = Description of Artificial Sequence: Synthetic primer
tgaaaagctccgggtcttagg

SEQ ID NO: 10 - DNA, 21 bp, synthetic construct
  source 1..21: mol_type = other DNA; note = Description of Artificial Sequence: Synthetic primer
ttgactggcgtgatgtagttg

SEQ ID NO: 11 - DNA, 21 bp, synthetic construct
  source 1..21: mol_type = other DNA; note = Description of Artificial Sequence: Synthetic primer
cttgggagacaacacgcattt

SEQ ID NO: 12 - DNA, 21 bp, synthetic construct
  source 1..21: mol_type = other DNA; note = Description of Artificial Sequence: Synthetic primer
tctcgctattcccgacctctt

SEQ ID NO: 13 - DNA, 19 bp, synthetic construct
  source 1..19: mol_type = other DNA; note = Description of Artificial Sequence: Synthetic primer
cagcgacctttccgtgtct

SEQ ID NO: 14 - DNA, 20 bp, synthetic construct
  source 1..20: mol_type = other DNA; note = Description of Artificial Sequence: Synthetic primer
gggtctccaatgctttggct

SEQ ID NO: 15 - DNA, 21 bp, synthetic construct
  source 1..21: mol_type = other DNA; note = Description of Artificial Sequence: Synthetic primer
gaggttggctctgactgtacc

SEQ ID NO: 16 - DNA, 21 bp, synthetic construct
  source 1..21: mol_type = other DNA; note = Description of Artificial Sequence: Synthetic primer
tccgtcccagtagattaccac

SEQ ID NO: 17 - DNA, 19 bp, synthetic construct
  source 1..19: mol_type = other DNA; note = Description of Artificial Sequence: Synthetic primer
cgggccatgcctgtttaca

SEQ ID NO: 18 - DNA, 21 bp, synthetic construct
  source 1..21: mol_type = other DNA; note = Description of Artificial Sequence: Synthetic primer
tgtccttcgttgaagtccctc

SEQ ID NO: 19 - DNA, 23 bp, synthetic construct
  source 1..23: mol_type = other DNA; note = Description of Artificial Sequence: Synthetic primer
gagtttgtcgctcctgagatagt

SEQ ID NO: 20 - DNA, 23 bp, synthetic construct
  source 1..23: mol_type = other DNA; note = Description of Artificial Sequence: Synthetic primer
gcttagtgtctccaagaaatggg

SEQ ID NO: 21 - DNA, 21 bp, synthetic construct
  source 1..21: mol_type = other DNA; note = Description of Artificial Sequence: Synthetic primer
tcccgcagagatactgactgt

SEQ ID NO: 22 - DNA, 21 bp, synthetic construct
  source 1..21: mol_type = other DNA; note = Description of Artificial Sequence: Synthetic primer
ggacccagaacacacctactc

SEQ ID NO: 23 - DNA, 21 bp, synthetic construct
  source 1..21: mol_type = other DNA; note = Description of Artificial Sequence: Synthetic primer
gtcatcgaacgcaccttccat

SEQ ID NO: 24 - DNA, 22 bp, synthetic construct
  source 1..22: mol_type = other DNA; note = Description of Artificial Sequence: Synthetic primer
agcttcaggtactcaaactcgt

SEQ ID NO: 25 - DNA, 20 bp, synthetic construct
  source 1..20: mol_type = other DNA; note = Description of Artificial Sequence: Synthetic primer
cccagagtttgagccgagtg

SEQ ID NO: 26 - DNA, 19 bp, synthetic construct
  source 1..19: mol_type = other DNA; note = Description of Artificial Sequence: Synthetic primer
cccatcccttcgtcgtcct

SEQ ID NO: 27 - DNA, 21 bp, synthetic construct
  source 1..21: mol_type = other DNA; note = Description of Artificial Sequence: Synthetic primer
atggtcaccttacctctgcaa

SEQ ID NO: 28 - DNA, 21 bp, synthetic construct
  source 1..21: mol_type = other DNA; note = Description of Artificial Sequence: Synthetic primer
tcatagcgtcggttgatgtcg

SEQ ID NO: 29 - DNA, 21 bp, synthetic construct
  source 1..21: mol_type = other DNA; note = Description of Artificial Sequence: Synthetic primer
cccgagaggtctttttccgag

SEQ ID NO: 30 - DNA, 21 bp, synthetic construct
  source 1..21: mol_type = other DNA; note = Description of Artificial Sequence: Synthetic primer
ccagcccatgatggttctgat

SEQ ID NO: 31 - DNA, 22 bp, synthetic construct
  source 1..22: mol_type = other DNA; note = Description of Artificial Sequence: Synthetic primer
taagttctgagtgtgaccgaga

SEQ ID NO: 32 - DNA, 22 bp, synthetic construct
  source 1..22: mol_type = other DNA; note = Description of Artificial Sequence: Synthetic primer
gctctgtctgtagggaggtagg

SEQ ID NO: 33 - DNA, 20 bp, synthetic construct
  source 1..20: mol_type = other DNA; note = Description of Artificial Sequence: Synthetic primer
atggaccgtagcatccctcc

SEQ ID NO: 34 - DNA, 20 bp, synthetic construct
  source 1..20: mol_type = other DNA; note = Description of Artificial Sequence: Synthetic primer
gtaggtgcgtaggttctggt

SEQ ID NO: 35 - DNA, 21 bp, synthetic construct
  source 1..21: mol_type = other DNA; note = Description of Artificial Sequence: Synthetic primer
gacctggaccctatggaggac

SEQ ID NO: 36 - DNA, 21 bp, synthetic construct
  source 1..21: mol_type = other DNA; note = Description of Artificial Sequence: Synthetic primer
cctcagtctggtcgtagatga

SEQ ID NO: 37 - DNA, 20 bp, synthetic construct
  source 1..20: mol_type = other DNA; note = Description of Artificial Sequence: Synthetic primer
cagggctcctgggtagaact

SEQ ID NO: 38 - DNA, 21 bp, synthetic construct
  source 1..21: mol_type = other DNA; note = Description of Artificial Sequence: Synthetic primer
ctactccgtccagactcatgc

SEQ ID NO: 39 - DNA, 23 bp, synthetic construct
  source 1..23: mol_type = other DNA; note = Description of Artificial Sequence: Synthetic primer
ttggatgcacaacatgaatcagg

SEQ ID NO: 40 - DNA, 23 bp, synthetic construct
  source 1..23: mol_type = other DNA; note = Description of Artificial Sequence: Synthetic primer
tcttctgactgagagctatggtc

SEQ ID NO: 41 - DNA, 21 bp, synthetic construct
  source 1..21: mol_type = other DNA; note = Description of Artificial Sequence: Synthetic primer
agaggtcataggggttccaca

SEQ ID NO: 42 - DNA, 21 bp, synthetic construct
  source 1..21: mol_type = other DNA; note = Description of Artificial Sequence: Synthetic primer
gctgacaacggtgtttccatt

SEQ ID NO: 43 - DNA, 21 bp, synthetic construct
  source 1..21: mol_type = other DNA; note = Description of Artificial Sequence: Synthetic primer
attccggtgatacacgagcag

SEQ ID NO: 44 - DNA, 19 bp, synthetic construct
  source 1..19: mol_type = other DNA; note = Description of Artificial Sequence: Synthetic primer
gctggtgggtctggtactc

SEQ ID NO: 45 - DNA, 22 bp, synthetic construct
  source 1..22: mol_type = other DNA; note = Description of Artificial Sequence: Synthetic primer
tctggacacccttgttgaatct

SEQ ID NO: 46 - DNA, 23 bp, synthetic construct
  source 1..23: mol_type = other DNA; note = Description of Artificial Sequence: Synthetic primer
tggaaaaggttcacaactgctac

SEQ ID NO: 47 - DNA, 19 bp, synthetic construct
  source 1..19: mol_type = other DNA; note = Description of Artificial Sequence: Synthetic primer
ggtggggtcatgtgtgtgg

SEQ ID NO: 48 - DNA, 22 bp, synthetic construct
  source 1..22: mol_type = other DNA; note = Description of Artificial Sequence: Synthetic primer
cggttcaggtactcagtcatcc

SEQ ID NO: 49 - DNA, 21 bp, synthetic construct
  source 1..21: mol_type = other DNA; note = Description of Artificial Sequence: Synthetic primer
tacaggctggctcaggactat

SEQ ID NO: 50 - DNA, 22 bp, synthetic construct
  source 1..22: mol_type = other DNA; note = Description of Artificial Sequence: Synthetic primer
cgcaacattttgtagcactctg

SEQ ID NO: 51 - DNA, 21 bp, synthetic construct
  source 1..21: mol_type = other DNA; note = Description of Artificial Sequence: Synthetic primer
gagctggtggttgactttctc

SEQ ID NO: 52 - DNA, 21 bp, synthetic construct
  source 1..21: mol_type = other DNA; note = Description of Artificial Sequence: Synthetic primer
tccatctccgattcagtccct

SEQ ID NO: 53 - DNA, 21 bp, synthetic construct
  source 1..21: mol_type = other DNA; note = Description of Artificial Sequence: Synthetic primer
gcggagttcacagctctatac

SEQ ID NO: 54 - DNA, 21 bp, synthetic construct
  source 1..21: mol_type = other DNA; note = Description of Artificial Sequence: Synthetic primer
aaaaggcccctacagttacca

SEQ ID NO: 55 - DNA, 23 bp, synthetic construct
  source 1..23: mol_type = other DNA; note = Description of Artificial Sequence: Synthetic primer
ttcctttagctcctaacacaggc

SEQ ID NO: 56 - DNA, 21 bp, synthetic construct
  source 1..21: mol_type = other DNA; note = Description of Artificial Sequence: Synthetic primer
tgctgctccagtgatagacga

SEQ ID NO: 57 - DNA, 22 bp, synthetic construct
  source 1..22: mol_type = other DNA; note = Description of Artificial Sequence: Synthetic primer
gttcagtggttcttactccagc

SEQ ID NO: 58 - DNA, 22 bp, synthetic construct
  source 1..22: mol_type = other DNA; note = Description of Artificial Sequence: Synthetic primer
actgtaggggttagtcctcgat

SEQ ID NO: 59 - DNA, 23 bp, synthetic construct
  source 1..23: mol_type = other DNA; note = Description of Artificial Sequence: Synthetic primer
tttccgtggctcttattcaaact

SEQ ID NO: 60 - DNA, 23 bp, synthetic construct
  source 1..23: mol_type = other DNA; note = Description of Artificial Sequence: Synthetic primer
gcacagtggtaggaacttctcat

SEQ ID NO: 61 - DNA, 22 bp, synthetic construct
  source 1..22: mol_type = other DNA; note = Description of Artificial Sequence: Synthetic primer
tagtgtatgcctcgtttgttgg

SEQ ID NO: 62 - DNA, 21 bp, synthetic construct
  source 1..21: mol_type = other DNA; note = Description of Artificial Sequence: Synthetic primer
ttctgtgtgtgctcacctttc

SEQ ID NO: 63 - DNA, 21 bp, synthetic construct
  source 1..21: mol_type = other DNA; note = Description of Artificial Sequence: Synthetic primer
tggctccctggattatcaaga

SEQ ID NO: 64 - DNA, 21 bp, synthetic construct
  source 1..21: mol_type = other DNA; note = Description of Artificial Sequence: Synthetic primer
ccagtgtcaggctaatgtctg

SEQ ID NO: 65 - DNA, 22 bp, synthetic construct
  source 1..22: mol_type = other DNA; note = Description of Artificial Sequence: Synthetic primer
tggggaacggaactgatttttc

SEQ ID NO: 66 - DNA, 21 bp, synthetic construct
  source 1..21: mol_type = other DNA; note = Description of Artificial Sequence: Synthetic primer
ttttgtggtgacttggggttg

SEQ ID NO: 67 - DNA, 21 bp, synthetic construct
  source 1..21: mol_type = other DNA; note = Description of Artificial Sequence: Synthetic primer
gagcaacaacttggtaaccct

SEQ ID NO: 68 - DNA, 22 bp, synthetic construct
  source 1..22: mol_type = other DNA; note = Description of Artificial Sequence: Synthetic primer
ggctggctataaatggagcttg

SEQ ID NO: 69 - DNA, 21 bp, synthetic construct
  source 1..21: mol_type = other DNA; note = Description of Artificial Sequence: Synthetic primer
ggacctccagatgcttgttga

SEQ ID NO: 70 - DNA, 19 bp, synthetic construct
  source 1..19: mol_type = other DNA; note = Description of Artificial Sequence: Synthetic primer
ggagctgcttggactgacg

SEQ ID NO: 71 - DNA, 21 bp, synthetic construct
  source 1..21: mol_type = other DNA; note = Description of Artificial Sequence: Synthetic primer
tctggttcttacgtctgttgc

SEQ ID NO: 72 - DNA, 21 bp, synthetic construct
  source 1..21: mol_type = other DNA; note = Description of Artificial Sequence: Synthetic primer
ctgtgcagtccctagctttcc

SEQ ID NO: 73 - DNA, 23 bp, synthetic construct
  source 1..23: mol_type = other DNA; note = Description of Artificial Sequence: Synthetic primer
aggatattgggctttacaacctg

SEQ ID NO: 74 - DNA, 23 bp, synthetic construct
  source 1..23: mol_type = other DNA; note = Description of Artificial Sequence: Synthetic primer
gaggtaacagaggtcagcatttt

SEQ ID NO: 75 - DNA, 21 bp, synthetic construct
  source 1..21: mol_type = other DNA; note = Description of Artificial Sequence: Synthetic primer
gtgcctttgtggctaaacact

SEQ ID NO: 76 - DNA, 21 bp, synthetic construct
  source 1..21: mol_type = other DNA; note = Description of Artificial Sequence: Synthetic primer
agtcccgttttgtccttacga

SEQ ID NO: 77 - DNA, 23 bp, synthetic construct
  source 1..23: mol_type = other DNA; note = Description of Artificial Sequence: Synthetic primer
aagggatttgttgacataacggg

SEQ ID NO: 78 - DNA, 21 bp, synthetic construct
  source 1..21: mol_type = other DNA; note = Description of Artificial Sequence: Synthetic primer
cagccgtagttcttcgtaagc

SEQ ID NO: 79 - DNA, 19 bp, synthetic construct
  source 1..19: mol_type = other DNA; note = Description of Artificial Sequence: Synthetic primer
gaccgcagctatgaccctc

SEQ ID NO: 80 - DNA, 21 bp, synthetic construct
  source 1..21: mol_type = other DNA; note = Description of Artificial Sequence: Synthetic primer
ctccggttcagcctctttaga

SEQ ID NO: 81 - DNA, 22 bp, synthetic construct
  source 1..22: mol_type = other DNA; note = Description of Artificial Sequence: Synthetic primer
tttccgcaaggttcgattttca

SEQ ID NO: 82 - DNA, 20 bp, synthetic construct
  source 1..20: mol_type = other DNA; note = Description of Artificial Sequence: Synthetic primer
ggcatctgcgctctaccatc

SEQ ID NO: 83 - DNA, 22 bp, synthetic construct
  source 1..22: mol_type = other DNA; note = Description of Artificial Sequence: Synthetic primer
agaaacctgctctacgaactgt

SEQ ID NO: 84 - DNA, 21 bp, synthetic construct
  source 1..21: mol_type = other DNA; note = Description of Artificial Sequence: Synthetic primer
gggaagcgagtctttcagaag

SEQ ID NO: 85 - DNA, 21 bp, synthetic construct
  source 1..21: mol_type = other DNA; note = Description of Artificial Sequence: Synthetic primer
accatgaaaagagaccccact

SEQ ID NO: 86 - DNA, 21 bp, synthetic construct
  source 1..21: mol_type = other DNA; note = Description of Artificial Sequence: Synthetic primer
gagtaccacgaaggcacaact

SEQ ID NO: 87 - DNA, 21 bp, synthetic construct
  source 1..21: mol_type = other DNA; note = Description of Artificial Sequence: Synthetic primer
agctgttgttgagcgaattgt

SEQ ID NO: 88 - DNA, 21 bp, synthetic construct
  source 1..21: mol_type = other DNA; note = Description of Artificial Sequence: Synthetic primer
agcaagttgaggagttccaca

SEQ ID NO: 89 - DNA, 23 bp, synthetic construct
  source 1..23: mol_type = other DNA; note = Description of Artificial Sequence: Synthetic primer
gaaattgtggaattgatgcgtga

SEQ ID NO: 90 - DNA, 23 bp, synthetic construct
  source 1..23: mol_type = other DNA; note = Description of Artificial Sequence: Synthetic primer
ctacaacgatcccctctgaaaaa

SEQ ID NO: 91 - DNA, 21 bp, synthetic construct
  source 1..21: mol_type = other DNA; note = Description of Artificial Sequence: Synthetic primer
caagagaagcaacgtatggca

SEQ ID NO: 92 - DNA, 22 bp, synthetic construct
  source 1..22: mol_type = other DNA; note = Description of Artificial Sequence: Synthetic primer
aggcagatggtcaaactctgta

SEQ ID NO: 93 - DNA, 22 bp, synthetic construct
  source 1..22: mol_type = other DNA; note = Description of Artificial Sequence: Synthetic primer
ttcaacaccacataacgtgtcc

SEQ ID NO: 94 - DNA, 22 bp, synthetic construct
  source 1..22: mol_type = other DNA; note = Description of Artificial Sequence: Synthetic primer
gtcaaggttgctcgttctatgg

SEQ ID NO: 95 - DNA, 23 bp, synthetic construct
  source 1..23: mol_type = other DNA; note = Description of Artificial Sequence: Synthetic primer
caccaacataactgaggtggatg

SEQ ID NO: 96 - DNA, 21 bp, synthetic construct
  source 1..21: mol_type = other DNA; note = Description of Artificial Sequence: Synthetic primer
aggaggagccatattttccca

SEQ ID NO: 97 - DNA, 20 bp, synthetic construct
  source 1..20: mol_type = other DNA; note = Description of Artificial Sequence: Synthetic primer
agtgacaggtatgggcgttc

SEQ ID NO: 98 - DNA, 21 bp, synthetic construct
  source 1..21: mol_type = other DNA; note = Description of Artificial Sequence: Synthetic primer
cggcatttgtatggtcctctt

SEQ ID NO: 99 - DNA, 22 bp, synthetic construct
  source 1..22: mol_type = other DNA; note = Description of Artificial Sequence: Synthetic primer
tcatggaccacagtaacatgga

SEQ ID NO: 100 - DNA, 23 bp, synthetic construct
  source 1..23: mol_type = other DNA; note = Description of Artificial Sequence: Synthetic primer
agtgaactgagatgtcagctcat

SEQ ID NO: 101 - DNA, 22 bp, synthetic construct
  source 1..22: mol_type = other DNA; note = Description of Artificial Sequence: Synthetic primer
ctcagaccagagattcgcaaac

SEQ ID NO: 102 - DNA, 22 bp, synthetic construct
  source 1..22: mol_type = other DNA; note = Description of Artificial Sequence: Synthetic primer
gcatttcccctcaaactctcaa

SEQ ID NO: 103 - DNA, 21 bp, synthetic construct
  source 1..21: mol_type = other DNA; note = Description of Artificial Sequence: Synthetic primer
aggagcagggacaagttacag

SEQ ID NO: 104 - DNA, 21 bp, synthetic construct
  source 1..21: mol_type = other DNA; note = Description of Artificial Sequence: Synthetic primer
ggacaatgggcatagggtgtt

SEQ ID NO: 105 - DNA, 20 bp, synthetic construct
  source 1..20: mol_type = other DNA; note = Description of Artificial Sequence: Synthetic primer
tggatgctctgtacgggaag

SEQ ID NO: 106 - DNA, 21 bp, synthetic construct
  source 1..21: mol_type = other DNA; note = Description of Artificial Sequence: Synthetic primer
ccaggctggtgtgaaactgaa

SEQ ID NO: 107 - DNA, 20 bp, synthetic construct
  source 1..20: mol_type = other DNA; note = Description of Artificial Sequence: Synthetic primer
ttccagcgatggcatgttcc

SEQ ID NO: 108 - DNA, 22 bp, synthetic construct
  source 1..22: mol_type = other DNA; note = Description of Artificial Sequence: Synthetic primer
tcctactgttgataagcccaca

SEQ ID NO: 109 - DNA, 20 bp, synthetic construct
  source 1..20: mol_type = other DNA; note = Description of Artificial Sequence: Synthetic primer
aaggtggagtaccacagagg

SEQ ID NO: 110 - DNA, 21 bp, synthetic construct
  source 1..21: mol_type = other DNA; note = Description of Artificial Sequence: Synthetic primer
tccatgtatggtgacccatcc

SEQ ID NO: 111 - DNA, 20 bp, synthetic construct
  source 1..20: mol_type = other DNA; note = Description of Artificial Sequence: Synthetic primer
gccaggttacggcagattca

SEQ ID NO: 112 - DNA, 19 bp, synthetic construct
  source 1..19: mol_type = other DNA; note = Description of Artificial Sequence: Synthetic primer
gaaggtcacgagcgtcacc

SEQ ID NO: 113 - DNA, 22 bp, synthetic construct
  source 1..22: mol_type = other DNA; note = Description of Artificial Sequence: Synthetic primer
actgaaaagcaatcgggaactt

SEQ ID NO: 114 - DNA, 21 bp, synthetic construct
  source 1..21: mol_type = other DNA; note = Description of Artificial Sequence: Synthetic primer
cacacacaatctccgcatctt

SEQ ID NO: 115 - DNA, 20 bp, synthetic construct
  source 1..20: mol_type = other DNA; note = Description of Artificial Sequence: Synthetic primer
cagagaggcactactgggct

SEQ ID NO: 116 - DNA, 21 bp, synthetic construct
  source 1..21: mol_type = other DNA; note = Description of Artificial Sequence: Synthetic primer
cggtatgcaaggatcacactg

SEQ ID NO: 117 - DNA, 21 bp, synthetic construct
  source 1..21: mol_type = other DNA; note = Description of Artificial Sequence: Synthetic primer
aatagtgccacgcagtctaca

SEQ ID NO: 118 - DNA, 21 bp, synthetic construct
  source 1..21: mol_type = other DNA; note = Description of Artificial Sequence: Synthetic primer
cagatggcctgtctaaggcaa

SEQ ID NO: 119 - DNA, 21 bp, synthetic construct
  source 1..21: mol_type = other DNA; note = Description of Artificial Sequence: Synthetic primer
ggtgggccaaaggatgaagag

SEQ ID NO: 120 - DNA, 20 bp, synthetic construct
  source 1..20: mol_type = other DNA; note = Description of Artificial Sequence: Synthetic primer
ccacaagccaaacgacttcc

SEQ ID NO: 121 - DNA, 21 bp, synthetic construct
  source 1..21: mol_type = other DNA; note = Description of Artificial Sequence: Synthetic primer
ggaagccatcaaacgtgactt

SEQ ID NO: 122 - DNA, 23 bp, synthetic construct
  source 1..23: mol_type = other DNA; note = Description of Artificial Sequence: Synthetic primer
cccgttccttattgaaaccaagc

SEQ ID NO: 123 - DNA, 20 bp, synthetic construct
  source 1..20: mol_type = other DNA; note = Description of Artificial Sequence: Synthetic primer
atgctggcgctactgtgttc

SEQ ID NO: 124 - DNA, 19 bp, synthetic construct
  source 1..19: mol_type = other DNA; note = Description of Artificial Sequence: Synthetic primer
ctccgccgagtcagagttg

SEQ ID NO: 125 - DNA, 21 bp, synthetic construct
  source 1..21: mol_type = other DNA; note = Description of Artificial Sequence: Synthetic primer
atggccctcaaacgtgtttac

SEQ ID NO: 126 - DNA, 19 bp, synthetic construct
  source 1..19: mol_type = other DNA; note = Description of Artificial Sequence: Synthetic primer
gcactggcgaaccttctca

SEQ ID NO: 127 - DNA, 21 bp, synthetic construct
  source 1..21: mol_type = other DNA; note = Description of Artificial Sequence: Synthetic primer
atcgagggaactattgacggc

SEQ ID NO: 128 - DNA, 22 bp, synthetic construct
  source 1..22: mol_type = other DNA; note = Description of Artificial Sequence: Synthetic primer
agttgtttgtaaggtccccgta

SEQ ID NO: 129 - DNA, 19 bp, synthetic construct
  source 1..19: mol_type = other DNA; note = Description of Artificial Sequence: Synthetic primer
gagaccccatgttcgaccc

SEQ ID NO: 130 - DNA, 22 bp, synthetic construct
  source 1..22: mol_type = other DNA; note = Description of Artificial Sequence: Synthetic primer
ggaatgcgaggaaccatagatg

SEQ ID NO: 131 - DNA, 20 bp, synthetic construct
  source 1..20: mol_type = other DNA; note = Description of Artificial Sequence: Synthetic primer
acggtgtgtatcaggctctg

SEQ ID NO: 132 - DNA, 22 bp, synthetic construct
  source 1..22: mol_type = other DNA; note = Description of Artificial Sequence: Synthetic primer
cacgtcggaaagaacatggtag

SEQ ID NO: 133 - DNA, 20 bp, synthetic construct
  source 1..20: mol_type = other DNA; note = Description of Artificial Sequence: Synthetic primer
acctcctgcaagaagagctg

SEQ ID NO: 134 - DNA, 20 bp, synthetic construct
  source 1..20: mol_type = other DNA; note = Description of Artificial Sequence: Synthetic primer
ctatctccacgtgctccaca

SEQ ID NO: 135 - DNA, 21 bp, synthetic construct
  source 1..21: mol_type = other DNA; note = Description of Artificial Sequence: Synthetic primer
ggggcgatcaatccagagaac

SEQ ID NO: 136 - DNA, 21 bp, synthetic construct
  source 1..21: mol_type = other DNA; note = Description of Artificial Sequence: Synthetic primer
gtactcggtaagtgtgccctg

SEQ ID NO: 137 - DNA, 22 bp, synthetic construct
  source 1..22: mol_type = other DNA; note = Description of Artificial Sequence: Synthetic primer
ccagaagcattaaccgagacaa

SEQ ID NO: 138 - DNA, 20 bp, synthetic construct
  source 1..20: mol_type = other DNA; note = Description of Artificial Sequence: Synthetic primer
cctcgaagctgaatcaaggc

SEQ ID NO: 139 - DNA, 23 bp, synthetic construct
  source 1..23: mol_type = other DNA; note = Description of Artificial Sequence: Synthetic primer
ttcagtatcacaacctcagcaag

SEQ ID NO: 140 - DNA, 22 bp, synthetic construct
  source 1..22: mol_type = other DNA; note = Description of Artificial Sequence: Synthetic primer
tggacctgcaagttaaaatccc

SEQ ID NO: 141 - DNA, 21 bp, synthetic construct
  source 1..21: mol_type = other DNA; note = Description of Artificial Sequence: Synthetic primer
cagatgttggggctaggattg

SEQ ID NO: 142 - DNA, 20 bp, synthetic construct
  source 1..20: mol_type = other DNA; note = Description of Artificial Sequence: Synthetic primer
gagtgttcggcacatgggta

SEQ ID NO: 143 - DNA, 23 bp, synthetic construct
  source 1..23: mol_type = other DNA; note = Description of Artificial Sequence: Synthetic primer
actcagcagtttaagaccattgc

SEQ ID NO: 144 - DNA, 21 bp, synthetic construct
  source 1..21: mol_type = other DNA; note = Description of Artificial Sequence: Synthetic primer
ggactctttcacatgcagagc

SEQ ID NO: 145 - DNA, 21 bp, synthetic construct
  source 1..21: mol_type = other DNA; note = Description of Artificial Sequence: Synthetic primer
ggaggtggtcggagataccaa

SEQ ID NO: 146 - DNA, 22 bp, synthetic construct
  source 1..22: mol_type = other DNA; note = Description of Artificial Sequence: Synthetic primer
acaatggcattacgagcaacat

SEQ ID NO: 147 - DNA, 21 bp, synthetic construct
  source 1..21: mol_type = other DNA; note = Description of Artificial Sequence: Synthetic primer
atgcctgtctgattcccatct

SEQ ID NO: 148 - DNA, 21 bp, synthetic construct
  source 1..21: mol_type = other DNA; note = Description of Artificial Sequence: Synthetic primer
catgacacttggcaatcctct

SEQ ID NO: 149 - DNA, 21 bp, synthetic construct
  source 1..21: mol_type = other DNA; note = Description of Artificial Sequence: Synthetic primer
tgagtctggacggagtagctc

SEQ ID NO: 150 - DNA, 22 bp, synthetic construct
  source 1..22: mol_type = other DNA; note = Description of Artificial Sequence: Synthetic primer
ccctgttggtatcttgtggtgt

SEQ ID NO: 151 - DNA, 21 bp, synthetic construct
  source 1..21: mol_type = other DNA; note = Description of Artificial Sequence: Synthetic primer
gacatcggggctaatctgaag

SEQ ID NO: 152 - DNA, 21 bp, synthetic construct
  source 1..21: mol_type = other DNA; note = Description of Artificial Sequence: Synthetic primer
ggctttactgtcacgtaccca

SEQ ID NO: 153 - DNA, 21 bp, synthetic construct
  source 1..21: mol_type = other DNA; note = Description of Artificial Sequence: Synthetic primer
ctttgccccgtgttttctagg

SEQ ID NO: 154 - DNA, 22 bp, synthetic construct
  source 1..22: mol_type = other DNA; note = Description of Artificial Sequence: Synthetic primer
ggataatcccgctgtagtttgg

SEQ ID NO: 155 - DNA, 22 bp, synthetic construct
  source 1..22: mol_type = other DNA; note = Description of Artificial Sequence: Synthetic primer
ccttacagggacaagtacagca

SEQ ID NO: 156 - DNA, 21 bp, synthetic construct
  source 1..21: mol_type = other DNA; note = Description of Artificial Sequence: Synthetic primer
ctgtgcctccgtaaatggttt

SEQ ID NO: 157 - DNA, 20 bp, synthetic construct
  source 1..20: mol_type = other DNA; note = Description of Artificial Sequence: Synthetic primer
gttgctggtcacattcctgg

SEQ ID NO: 158 - DNA, 21 bp, synthetic construct
  source 1..21: mol_type = other DNA; note = Description of Artificial Sequence: Synthetic primer
gcaggtaatcccaaaagcgac

SEQ ID NO: 159 - DNA, 20 bp, synthetic construct
  source 1..20: mol_type = other DNA; note = Description of Artificial Sequence: Synthetic primer
gtgctgaagctgtctctcgg

SEQ ID NO: 160 - DNA, 23 bp, synthetic construct
  source 1..23: mol_type = other DNA; note = Description of Artificial Sequence: Synthetic primer
gcccaaggtaggaaacagtcttt

SEQ ID NO: 161 - DNA, 21 bp, synthetic construct
  source 1..21: mol_type = other DNA; note = Description of Artificial Sequence: Synthetic primer
tggagctggtaacccagtagg

SEQ ID NO: 162 - DNA, 23 bp, synthetic construct
  source 1..23: mol_type = other DNA; note = Description of Artificial Sequence: Synthetic primer
cctttgccttggagtatttggta

SEQ ID NO: 163 - DNA, 20 bp, synthetic construct
  source 1..20: mol_type = other DNA; note = Description of Artificial Sequence: Synthetic primer
ccagcagtcgtctttgtcac

SEQ ID NO: 164 - DNA, 20 bp, synthetic construct
  source 1..20: mol_type = other DNA; note = Description of Artificial Sequence: Synthetic primer
ctctgggttggcacacactt

SEQ ID NO: 165 - DNA, 19 bp, synthetic construct
  source 1..19: mol_type = other DNA; note = Description of Artificial Sequence: Synthetic primer
cccggctctatgccaactg

SEQ ID NO: 166 - DNA, 21 bp, synthetic construct
  source 1..21: mol_type = other DNA; note = Description of Artificial Sequence: Synthetic primer
ccatgtgcttgaactggctga

SEQ ID NO: 167 - DNA, 21 bp, synthetic construct
  source 1..21: mol_type = other DNA; note = Description of Artificial Sequence: Synthetic primer
cactgtctcactacgtgtcgg

SEQ ID NO: 168 - DNA, 19 bp, synthetic construct
  source 1..19: mol_type = other DNA; note = Description of Artificial Sequence: Synthetic primer
ccagccaatggaggtcagc

SEQ ID NO: 169 - DNA, 22 bp, synthetic construct
  source 1..22: mol_type = other DNA; note = Description of Artificial Sequence: Synthetic primer
gccttgcttaccttatgaggac

SEQ ID NO: 170 - DNA, 20 bp, synthetic construct
  source 1..20: mol_type = other DNA; note = Description of Artificial Sequence: Synthetic primer
gggagagatgatgcttcgcc

SEQ ID NO: 171 - DNA, 20 bp, synthetic construct
  source 1..20: mol_type = other DNA; note = Description of Artificial Sequence: Synthetic primer
ccaaccagattgtgcgcttc

SEQ ID NO: 172 - DNA, 19 bp, synthetic construct
  source 1..19: mol_type = other DNA; note = Description of Artificial Sequence: Synthetic primer
cgcaggtcctctcacagtc

SEQ ID NO: 173 - DNA, 21 bp, synthetic construct
  source 1..21: mol_type = other DNA; note = Description of Artificial Sequence: Synthetic primer
atacgtggattgaggaccact

SEQ ID NO: 174 - DNA, 21 bp, synthetic construct
  source 1..21: mol_type = other DNA; note = Description of Artificial Sequence: Synthetic primer
tccaatgtcaagtagcggttg

SEQ ID NO: 175 - DNA, 22 bp, synthetic construct
  source 1..22: mol_type = other DNA; note = Description of Artificial Sequence: Synthetic primer
tcctacgtttacctgtccatgt

SEQ ID NO: 176 - DNA, 22 bp, synthetic construct
  source 1..22: mol_type = other DNA; note = Description of Artificial Sequence: Synthetic primer
gtttgtgcagttccagtagtga

SEQ ID NO: 177 - DNA, 21 bp, synthetic construct
  source 1..21: mol_type = other DNA; note = Description of Artificial Sequence: Synthetic primer
ggcacaaggacgttctcaagt

SEQ ID NO: 178 - DNA, 19 bp, synthetic construct
  source 1..19: mol_type = other DNA; note = Description of Artificial Sequence: Synthetic primer
cagacaggaccaaccggac

SEQ ID NO: 179 - DNA, 23 bp, synthetic construct
  source 1..23: mol_type = other DNA; note = Description of Artificial Sequence: Synthetic primer
tgtcttggaatgcactgtatctc

SEQ ID NO: 180 - DNA, 22 bp, synthetic construct
  source 1..22: mol_type = other DNA; note = Description of Artificial Sequence: Synthetic primer
cccagtaaggctgtaaatgctc

SEQ ID NO: 181 - DNA, 21 bp, synthetic construct
  source 1..21: mol_type = other DNA; note = Description of Artificial Sequence: Synthetic primer
cagtcggtgtatgccttctcg

SEQ ID NO: 182 - DNA, 19 bp, synthetic construct
  source 1..19: mol_type = other DNA; note = Description of Artificial Sequence: Synthetic primer
gagggacgccacattctcg

SEQ ID NO: 183 - DNA, 20 bp, synthetic construct
  source 1..20: mol_type = other DNA; note = Description of Artificial Sequence: Synthetic primer
gaatgggcagaacgagcatc

SEQ ID NO: 184 - DNA, 20 bp, synthetic construct
  source 1..20: mol_type = other DNA; note = Description of Artificial Sequence: Synthetic primer
ccggccctatgaggaacttc

SEQ ID NO: 185 - DNA, 20 bp, synthetic construct
  source 1..20: mol_type = other DNA; note = Description of Artificial Sequence: Synthetic primer
gagcttgcaccattcggtct

SEQ ID NO: 186 - DNA, 23 bp, synthetic construct
  source 1..23: mol_type = other DNA; note = Description of Artificial Sequence: Synthetic primer
gggtaggaaggatctctgagttc

SEQ ID NO: 187 - DNA, 23 bp, synthetic construct
  source 1..23: mol_type = other DNA; note = Description of Artificial Sequence: Synthetic primer
gaggcaagaccgaagtaaactac

SEQ ID NO: 188 - DNA, 20 bp, synthetic construct
  source 1..20: mol_type = other DNA; note = Description of Artificial Sequence: Synthetic primer
ccgaactggttacacgggaa

SEQ ID NO: 189 - DNA, 23 bp, synthetic construct
  source 1..23: mol_type = other DNA; note = Description of Artificial Sequence: Synthetic primer
atgactacacagttaagggtcgt

SEQ ID NO: 190 - DNA, 21 bp, synthetic construct
  source 1..21: mol_type = other DNA; note = Description of Artificial Sequence: Synthetic primer
ggatattgcgctgtcagacca

SEQ ID NO: 191 - DNA, 21 bp, synthetic construct
  source 1..21: mol_type = other DNA; note = Description of Artificial Sequence: Synthetic primer
caaaggggtaacaggcaccat

SEQ ID NO: 192 - DNA, 19 bp, synthetic construct
  source 1..19: mol_type = other DNA; note = Description of Artificial Sequence: Synthetic primer
ggcggccacattgacaaac

SEQ ID NO: 193 - DNA, 21 bp, synthetic construct
  source 1..21: mol_type = other DNA; note = Description of Artificial Sequence: Synthetic primer
cgcacctacagtgtctcattc

SEQ ID NO: 194 - DNA, 21 bp, synthetic construct
  source 1..21: mol_type = other DNA; note = Description of Artificial Sequence: Synthetic primer
caggtacttgaaggcaggatg

SEQ ID NO: 195 - DNA, 21 bp, synthetic construct
  source 1..21: mol_type = other DNA; note = Description of Artificial Sequence: Synthetic primer
cacttgcgtgaatgttggatg

SEQ ID NO: 196 - DNA, 20 bp, synthetic construct
  source 1..20: mol_type = other DNA; note = Description of Artificial Sequence: Synthetic primer
tgggatcactcgtgaaggct

SEQ ID NO: 197 - DNA, 21 bp, synthetic construct
  source 1..21: mol_type = other DNA; note = Description of Artificial Sequence: Synthetic primer
ggaacatccatgtgatccgac

SEQ ID NO: 198 - DNA, 20 bp, synthetic construct
  source 1..20: mol_type = other DNA; note = Description of Artificial Sequence: Synthetic primer
gccatcccggaagtaaacca

SEQ ID NO: 199 - DNA, 20 bp, synthetic construct
  source 1..20: mol_type = other DNA; note = Description of Artificial Sequence: Synthetic primer
cgacctgaacaagagcatca

SEQ ID NO: 200 - DNA, 20 bp, synthetic construct
  source 1..20: mol_type = other DNA; note = Description of Artificial Sequence: Synthetic primer
aagatctgcgtctgcttggt

SEQ ID NO: 201 - DNA, 21 bp, synthetic construct
  source 1..21: mol_type = other DNA; note = Description of Artificial Sequence: Synthetic primer
agagtggaccaactgaagagt

SEQ ID NO: 202 - DNA, 21 bp, synthetic construct
  source 1..21: mol_type = other DNA; note = Description of Artificial Sequence: Synthetic primer
attctctgcatttgtccgctt

SEQ ID NO: 203 - DNA, 21 bp, synthetic construct
  source 1..21: mol_type = other DNA; note = Description of Artificial Sequence: Synthetic primer
ctctttctccgcgagcataac

SEQ ID NO: 204 - DNA, 23 bp, synthetic construct
  source 1..23: mol_type = other DNA; note = Description of Artificial Sequence: Synthetic primer
aatgggtaggtagtccctaaagg

SEQ ID NO: 205 - DNA, 21 bp, synthetic construct
  source 1..21: mol_type = other DNA; note = Description of Artificial Sequence: Synthetic primer
ccctgtttccatcactccctc

SEQ ID NO: 206 - DNA, 19 bp, synthetic construct
  source 1..19: mol_type = other DNA; note = Description of Artificial Sequence: Synthetic primer
gcaggtcttttgggcatcc

SEQ ID NO: 207 - DNA, 21 bp, synthetic construct
  source 1..21: mol_type = other DNA; note = Description of Artificial Sequence: Synthetic primer
ccagatcatcacttaccggga

SEQ ID NO: 208 - DNA, 22 bp, synthetic construct
  source 1..22: mol_type = other DNA; note = Description of Artificial Sequence: Synthetic primer
cactgagtcattgtaggaacgg

SEQ ID NO: 209 - DNA, 19 bp, synthetic construct
  source 1..19: mol_type = other DNA; note = Description of Artificial Sequence: Synthetic primer
ggccatctacccgacctct

SEQ ID NO: 210 - DNA, 20 bp, synthetic construct
  source 1..20: mol_type = other DNA; note = Description of Artificial Sequence: Synthetic primer
gccattggggttcttgctca

SEQ ID NO: 211 - DNA, 22 bp, synthetic construct
  source 1..22: mol_type = other DNA; note = Description of Artificial Sequence: Synthetic primer
gaagagcactgatcgtactggc

SEQ ID NO: 212 - DNA, 21 bp, synthetic construct
  source 1..21: mol_type = other DNA; note = Description of Artificial Sequence: Synthetic primer
ggatactgaaagttcgcaggg

SEQ ID NO: 213 - DNA, 21 bp, synthetic construct
  source 1..21: mol_type = other DNA; note = Description of Artificial Sequence: Synthetic primer
gctcatggacgtgcatgtctt

SEQ ID NO: 214 - DNA, 21 bp, synthetic construct
  source 1..21: mol_type = other DNA; note = Description of Artificial Sequence: Synthetic primer
gtggaaaccagtagctgtcgt

SEQ ID NO: 215 - DNA, 22 bp, synthetic construct
  source 1..22: mol_type = other DNA; note = Description of Artificial Sequence: Synthetic primer
tagcatctgagccactgaaagt

SEQ ID NO: 216 - DNA, 22 bp, synthetic construct
  source 1..22: mol_type = other DNA; note = Description of Artificial Sequence: Synthetic primer
caccaccgaaagctagtgaatc

SEQ ID NO: 217 - DNA, 19 bp, synthetic construct
  source 1..19: mol_type = other DNA; note = Description of Artificial Sequence: Synthetic primer
agggacgattacgagctgc

SEQ ID NO: 218 - DNA, 22 bp, synthetic construct
  source 1..22: mol_type = other DNA; note = Description of Artificial Sequence: Synthetic primer
tccgtttgattgccactttctc

SEQ ID NO: 219 - DNA, 22 bp, synthetic construct
  source 1..22: mol_type = other DNA; note = Description of Artificial Sequence: Synthetic primer
gacacacttggaagctcagaac

SEQ ID NO: 220 - DNA, 20 bp, synthetic construct
  source 1..20: mol_type = other DNA; note = Description of Artificial Sequence: Synthetic primer
gtaaagggcatggcacttcg

SEQ ID NO: 221 - DNA, 19 bp, synthetic construct
  source 1..19: mol_type = other DNA; note = Description of Artificial Sequence: Synthetic primer
ctgacccaggttacggacc

SEQ ID NO: 222 - DNA, 19 bp, synthetic construct
  source 1..19: mol_type = other DNA; note = Description of Artificial Sequence: Synthetic primer
tcccggtagttgaggggtt

SEQ ID NO: 223 - DNA, 21 bp, synthetic construct
  source 1..21: mol_type = other DNA; note = Description of Artificial Sequence: Synthetic primer
gaagctgtcggactacaaagg

SEQ ID NO: 224 - DNA, 19 bp, synthetic construct
  source 1..19: mol_type = other DNA; note = Description of Artificial Sequence: Synthetic primer
tcggtggggcacacaaaag

SEQ ID NO: 225 - DNA, 19 bp, synthetic construct
  source 1..19: mol_type = other DNA; note = Description of Artificial Sequence: Synthetic primer
gcaagacggtgcagtgaag

SEQ ID NO: 226 - DNA, 21 bp, synthetic construct
  source 1..21: mol_type = other DNA; note = Description of Artificial Sequence: Synthetic primer
atggcatctcccaccttgatt

SEQ ID NO: 227 - DNA, 21 bp, synthetic construct
  source 1..21: mol_type = other DNA; note = Description of Artificial Sequence: Synthetic primer
gactcatggggcattctcttc

SEQ ID NO: 228 - DNA, 22 bp, synthetic construct
  source 1..22: mol_type = other DNA; note = Description of Artificial Sequence: Synthetic primer
caagctcccgattcctatcatc

SEQ ID NO: 229 - DNA, 20 bp, synthetic construct
  source 1..20: mol_type = other DNA; note = Description of Artificial Sequence: Synthetic primer
agtcagtggaacaagccgag

SEQ ID NO: 230 - DNA, 21 bp, synthetic construct
  source 1..21: mol_type = other DNA; note = Description of Artificial Sequence: Synthetic primer
ctgccgaaatgtatgatgggc

SEQ ID NO: 231 - DNA, 20 bp, synthetic construct
  source 1..20: mol_type = other DNA; note = Description of Artificial Sequence: Synthetic primer
cgggaggcgacaagatgttc

SEQ ID NO: 232 - DNA, 20 bp, synthetic construct
  source 1..20: mol_type = other DNA; note = Description of Artificial Sequence: Synthetic primer
tccccagaccacaacacagt

SEQ ID NO: 233 - DNA, 20 bp, synthetic construct
  source 1..20: mol_type = other DNA; note = Description of Artificial Sequence: Synthetic primer
tcacccaggagtgctacgat

SEQ ID NO: 234 - DNA, 20 bp, synthetic construct
  source 1..20: mol_type = other DNA; note = Description of Artificial Sequence: Synthetic primer
gagccgtgtgtagttctgcc

SEQ ID NO: 235 - DNA, 22 bp, synthetic construct
  source 1..22: mol_type = other DNA; note = Description of Artificial Sequence: Synthetic primer
gaagacctgcgagtaaaactga

SEQ ID NO: 236 - DNA, 23 bp, synthetic construct
  source 1..23: mol_type = other DNA; note = Description of Artificial Sequence: Synthetic primer
acaagacggccacatctatcata

SEQ ID NO: 237 - DNA, 22 bp, synthetic construct
  source 1..22: mol_type = other DNA; note = Description of Artificial Sequence: Synthetic primer
cacgcagaacatagataccctg

SEQ ID NO: 238 - DNA, 22 bp, synthetic construct
  source 1..22: mol_type = other DNA; note = Description of Artificial Sequence: Synthetic primer
cagtgtgatgtgtagaaggtgc

SEQ ID NO: 239 - DNA, 21 bp, synthetic construct
  source 1..21: mol_type = other DNA; note = Description of Artificial Sequence: Synthetic primer
gactacgacttgtgtagcgtc

SEQ ID NO: 240 - DNA, 21 bp, synthetic construct
  source 1..21: mol_type = other DNA; note = Description of Artificial Sequence: Synthetic primer
agtgtccgtgtttcaccttcc

SEQ ID NO: 241 - DNA, 23 bp, synthetic construct
  source 1..23: mol_type = other DNA; note = Description of Artificial Sequence: Synthetic primer
cagggaggtgactacttctactc

SEQ ID NO: 242 - DNA, 21 bp, synthetic construct
  source 1..21: mol_type = other DNA; note = Description of Artificial Sequence: Synthetic primer
caggtacacccttaggtctga

SEQ ID NO: 243 - DNA, 21 bp, synthetic construct
  source 1..21: mol_type = other DNA; note = Description of Artificial Sequence: Synthetic primer
gctcctacctaaagagcacca

SEQ ID NO: 244 - DNA, 22 bp, synthetic construct
  source 1..22: mol_type = other DNA; note = Description of Artificial Sequence: Synthetic primer
tggcaatgtatgtctcctccag

SEQ ID NO: 245 - DNA, 19 bp, synthetic construct
  source 1..19: mol_type = other DNA; note = Description of Artificial Sequence: Synthetic primer
ctgtcacgctggttatggc

SEQ ID NO: 246 - DNA, 22 bp, synthetic construct
  source 1..22: mol_type = other DNA; note = Description of Artificial Sequence: Synthetic primer
gctagagacacgagactcctca

SEQ ID NO: 247 - DNA, 20 bp, synthetic construct
  source 1..20: mol_type = other DNA; note = Description of Artificial Sequence: Synthetic primer
ccccatcgcccataagacac

SEQ ID NO: 248 - DNA, 20 bp, synthetic construct
  source 1..20: mol_type = other DNA; note = Description of Artificial Sequence: Synthetic primer
ccacgtagccctcttgcttc

SEQ ID NO: 249 - DNA, 21 bp, synthetic construct
  source 1..21: mol_type = other DNA; note = Description of Artificial Sequence: Synthetic primer
gtgaagcagatcgagagcaag

SEQ ID NO: 250 - DNA, 23 bp, synthetic construct
  source 1..23: mol_type = other DNA; note = Description of Artificial Sequence: Synthetic primer
cgtggctgagaagtcaactacta

SEQ ID NO: 251 - DNA, 23 bp, synthetic construct
  source 1..23: mol_type = other DNA; note = Description of Artificial Sequence: Synthetic primer
atgggcaatttattggtcctcac

SEQ ID NO: 252 - DNA, 22 bp, synthetic construct
  source 1..22: mol_type = other DNA; note = Description of Artificial Sequence: Synthetic primer
cccaagtaacgtggtctttcac

SEQ ID NO: 253 - DNA, 19 bp, synthetic construct
  source 1..19: mol_type = other DNA; note = Description of Artificial Sequence: Synthetic primer
cggcttcgaccagcaaatg

SEQ ID NO: 254 - DNA, 20 bp, synthetic construct
  source 1..20: mol_type = other DNA; note = Description of Artificial Sequence: Synthetic primer
acaggacggtgtcaaaggtg

SEQ ID NO: 255 - DNA, 21 bp, synthetic construct
  source 1..21: mol_type = other DNA; note = Description of Artificial Sequence: Synthetic primer
acctatggctggtacatcgtc

SEQ ID NO: 256 - DNA, 19 bp, synthetic construct
  source 1..19: mol_type = other DNA; note = Description of Artificial Sequence: Synthetic primer
gcctcaaggctcttagccg

SEQ ID NO: 257 - DNA, 19 bp, synthetic construct
  source 1..19: mol_type = other DNA; note = Description of Artificial Sequence: Synthetic primer
ttggtgctgaacgtctggg

SEQ ID NO: 258 - DNA, 21 bp, synthetic construct
  source 1..21: mol_type = other DNA; note = Description of Artificial Sequence: Synthetic primer
tgggtgacccttaaagagcct

(19) United States
(12) Patent Application Publication
(10) Pub. No.: US 2024/0408402 A1
(43) Pub. Date: Dec. 12, 2024

(54) METHOD FOR TREATMENT OF CANCER WITH COMBINATION OF COLD ATMOSPHERIC PLASMA AND A GENE INHIBITOR

(71) Applicant: Jerome Canady Research Institute for Advanced Biological and Technological Sciences, Takoma Park, MD (US)

(72) Inventors: Jerome Canady, Lakeland, FL (US); Saravana Murthy, Owings Mills, MD (US); Xiaoqian Cheng, Owings Mills, MD (US); Taisen Zhuang, Rockvillle, MD (US)

(21) Appl. No.: 18/782,803

(22) Filed: Jul. 24, 2024

Related U.S. Application Data
(63) Continuation of application No. PCT/US2023/012390, filed on Feb. 6, 2023.
(63) Continuation-in-part of application No. 17/135,883, filed on Dec. 28, 2020, now Pat. No. 12,048,704.
(60) Provisional application No. 63/306,541, filed on Feb. 4, 2022.
(60) Provisional application No. 62/953,754, filed on Dec. 26, 2019.

(51) Int. Cl. A61N 1/44; A61K 31/551; A61P 35/00

(52) U.S. Cl. CPC A61N 1/44; A61K 31/551; A61P 35/00

(57) ABSTRACT

A method for treating cancer. The method comprises treating a patient having a cancerous tumor with the gene inhibitor CPI203 pre-operatively to inhibit upregulation of a particular gene, surgically removing the cancerous tumor, treating the patient with the gene inhibitor intra-operatively to inhibit upregulation of a particular gene, applying cold atmospheric plasma to surgical margins around the area in the patient from which the tumor was surgically removed, and treating the patient with the gene inhibitor CPI203 post-operatively.

### CROSS-REFERENCE TO RELATED APPLICATIONS

The present application is a continuation of International Application No. PCT/US2023/012390 filed on Feb. 6, 2023, which claims the benefit of the filing date of U.S. Provisional Patent Application Ser. No. 63/306,541, and a continuation-in-part of U.S. patent application Ser. No. 17/135,883, filed on Dec. 28, 2020, which claims the benefit of the filing date of U.S. Provisional Patent Application Ser. No. 62/953,754, filed on Dec. 26, 2019.

The aforementioned patent applications are hereby incorporated by reference in their entirety.

### STATEMENT REGARDING FEDERALLY SPONSORED RESEARCH OR DEVELOPMENT

None.

### SEQUENCE LISTING

The instant application contains a Sequence Listing which has been submitted electronically and is hereby incorporated by reference in its entirety. Said electronic copy, created on Feb. 3, 2023, is named 9101_161PCT_SL.xml and is 278 bytes in size

### BACKGROUND OF THE INVENTION

### Field of the Invention

The present invention relates to systems and methods for treating cancer with cold atmospheric plasma.

### Brief Description of the Related Art

Breast cancer is the most common cause of cancer death among women worldwide (F. Bray, J. Ferlay, I. Soerjomataram, R. L. Siegel, L. A. Torre, A. Jemal, “Global cancer statistics 2018: GLOBOCAN estimates of incidence and mortality worldwide for 36 cancers in 185 countries,” CA Cancer J Clin, 68 (2018) 394-424) and it exhibit diverse molecular features that reflect the high heterogeneity which complicates the clinical treatment (N. Howlader, K. A. Cronin, A. W. Kurian, R. Andridge, “Differences in Breast Cancer Survival by Molecular Subtypes in the United States,” Cancer Epidemiol Biomarkers Prev, 27 (2018) 619-626). Breast cancers are categorized by the molecular receptor status that are expressed such as the estrogen receptor (ER), progesterone receptor (PR) and human epidermal growth factor receptor 2 (HER2) expression. Prognosis for breast cancer patients is generally favorable with ER+/PR+ tumors, intermediate with either ER+/PR− or ER−/PR+ tumors, and usually poor for ER−/PR− tumors. Based on the receptor status selective therapeutic interventions are carried out. For example, in the case of ER+ tumor estrogen-receptor modulators, such as Tamoxifen and letrozole are administered, trastuzumab is (Herceptin), a humanized monoclonal antibody developed to target and inhibit the function of HER2 and a dual anti-HER2 regimen, pertuzumab in combination with trastuzumab and docetaxel are administered to mitigate the risk of mortality to considerable effect, however the incidences of adverse events and resistance to these drugs are not uncommon. Triple negative breast cancer (TNBC) (ER−/PR−, HER2−) do not respond to endocrine therapy or HER2-targeted therapies. Therapies targeting TRAIL (TNF (tumor necrosis factor)-related apoptosis-inducing ligand) and cyclin dependent kinases (CDK) or cell cycle regulators have been used with limited success. In recent years Cold atmospheric plasma (CAP) technology that utilizes ionized gas to selectively induce apoptosis in cancer cells have shown very encouraging results. Preclinical In vivo studies in mouse models for various cancers for CAP treatment have demonstrated to effectively reduce tumor growth rate and induce cancer cell death. CAP treatment induces apoptosis in various breast cancer cells and the potency of the treatment depend on a combination of parameters such as the concentration and the time of the plasma treatment. Susceptibility or resistance to CAP treatment is also determined by the molecular features of the cell types such as the receptor status which are classified into intrinsic subtypes including luminal A (ER+PR+/−HER2−), luminal B (ER+PR+/−HER2+), basal-like (ER−PR−HER2−), and HER2-positive (ER−PR−HER2+). At high concentrations and duration of CAP treatment most of the breast cancer cells often undergo apoptosis due to the release of reactive oxygen and nitrogen species (RONS) and oxidative stress-induced cell toxicity of these species. The mechanism of such oxidative stress-induced cell death process is broadly discussed in various studies. CAP treatment on subtypes of breast cancer cell lines has been demonstrated to reduce breast cancer viability by 92-99% regardless of the status of the receptors on these cells at the most optimal power setting and time of treatment. However, some subset of cells resists the CAP insult and survive, but the molecular mechanism for such survival in these cells has not been systematically investigated.

Several different systems and methods for performing Cold Atmospheric Plasma (CAP) treatment have been disclosed. For example, U.S. Pat. No. 10,213,614 discloses a two-electrode system for CAP treatment. U.S. Pat. Nos. 9,999,462 and 10,023,858 each disclose a converter unit for using a traditional electrosurgical system with a single electrode CAP accessory to perform CAP treatment. WO 2018191265A1 disclosed an integrated electrosurgical generator and gas control module for performing CAP.

### SUMMARY OF THE INVENTION

Breast cancer is the leading cause of cancer death among women. Predominantly, the poor prognosis is due to the triple-negative breast cancer characterized by the absence or low-level expression of estrogen (ER), progesterone (ER), and HER2 receptors. Cold atmospheric plasma (CAP) jet delivered by the Canady Cold Plasma Conversion system that induces cell death in triple-negative breast cancer cell line without thermal damage, however, the mechanism of cell death by CAP treatment is ambiguous. In this study, we aimed to investigate the gene expression profile by screening the expressions of apoptotic and oxidative stress related gene markers in breast cancer cell lines after CAP treatment to determine the molecular mechanism of CAP induced cell death. Six different types of breast cancer cell lines including MCF-7 and T-47D (luminal A: ER+ PR+/−HER2−), BT-474 (luminal B: ER+PR+/−HER2+), SK-BR-3 (ER−PR−HER2+), MDA-MB-231 and Hs578T (basal-like: ER−PR−HER2−) were tested with Canady Helios Cold Plasma Scalpel (cold atmospheric plasma) with 2 power settings (80 p and 120 p, which are approximately 15 W, and 28 W respectively). Gene expression of 48 apoptotic and 35 oxidative gene markers were determined at 4 different time points (3 hrs, 6 hrs 12 hrs and 24 hrs) after treatment with CAP, using quantitative real time polymerase chain reaction (qRT-PCR). After CAP treatment, the expression level of BCL2A1 and TNF were significantly increased in triple-negative cell lines, MDA-MB-231 and Hs578T (p<0.01). In contrast, the HER2-positive and ER, PR positive cell lines showed little or no expression of BCL2A1 (p<0.01). Silencing BCL2A1 mRNA by siRNA increased the potency of the CAP treatment. Combination of CAP and CPI203, a BET bromodomain inhibitor dramatic increased the CAP-induced cell death without any cytotoxicity by itself (p<0.05). Our results revealed that BCL2A1, act as potential key gene in breast cancer survival, thereby improving our understanding of molecular profiles of CAP induced cell death. The upregulation of BCL2A1 mRNA levels and expression of protein only after CAP induction in triple negative subtype could correspond an important molecular mechanism for their poor prognostic significance which could be reversed with a combination of siRNA or BCL2A1 antagonist-CAP therapy.

In a preferred embodiment, the present invention is a method for treatment of cancer. The method comprises treating a patient having a cancerous tumor with a gene inhibitor pre-operatively to inhibit upregulation of a particular gene, surgically removing the cancerous tumor, treating the patient with the gene inhibitor intra-operatively to inhibit upregulation of a particular gene, applying cold atmospheric plasma to surgical margins around the area in the patient from which the tumor was surgically removed, and treating the patient with the gene inhibitor post-operatively. The cancer may be triple negative breast cancer and the particular survival gene may be BCL2A1. The gene inhibitor may be CPI203. The pre-operative treatment of a patient with a gene inhibitor is within 24 hours of surgical removal of the cancerous tumor. The post-operative treatment of a patient with a gene inhibitor is within 24 hours after surgical removal of the cancerous tumor.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1 is a flow diagram illustrating a method in accordance with a preferred embodiment of the present invention.

FIG. 2 is a perspective view of a preferred embodiment of a gas-enhanced electrosurgical generator that may be used in a preferred embodiment of the present invention.

FIG. 3 is a block diagram of a cold atmospheric plasma generator in accordance with a preferred embodiment of the present invention.

FIG. 4A is a block diagram of an embodiment of a cold atmospheric plasma system with an electrosurgical generator and a low frequency converter for producing cold plasma.

FIG. 4B is a block diagram of an embodiment of an integrated cold atmospheric plasma system that can perform multiple types of plasma surgeries.

FIG. 5 is perspective view of a cold atmospheric plasma probe that may be used in a preferred embodiment of the present invention.

FIG. 6A is an assembly view of a handpiece of a cold atmospheric plasma probe that may be used in a preferred embodiment of the present invention.

FIG. 6B is an assembly view of a cable harness of a cold atmospheric plasma probe that may be used in a preferred embodiment of the present invention.

FIG. 7 (Table 1) is a table of genes selected based on their ability in “induction of apoptosis.” Figure discloses SEQ ID NOS 1-22, respectively, in order of appearance.

FIGS. 8A-8B (Table 2) are a table of genes selected based on their ability in “regulation of apoptosis.” FIGS. 8A-8B disclose SEQ ID NOS 9-10, 23-68, 11-12, and 69-80, respectively, in order of appearance. FIG. 8B is a continuation of FIG. 8A.

FIGS. 9A-9B (Table 3) are a table of genes selected based on their ability as “caspases & regulators.” FIGS. 9A-9B disclose SEQ ID NOS 81-118, respectively, in order of appearance. FIG. 9B is a continuation of FIG. 9A.

FIGS. 10A-10F (Table 4) are a table of genes selected based on their ability as “responders of oxidative stress.” FIGS. 10A-10F disclose SEQ ID NOS 119-256, 165-166, and 257-258, respectively, in order of appearance.

FIG. 11 is a bar graph showing the differential gene expression of genes involved in the induction of apoptosis after CAP treatment in triple negative breast cancer cell line MDA-MB-231.

FIGS. 12A-12B are bar graphs showing the differential gene expression of genes involved in the positive (FIG. 12A) and negative (FIG. 12B) regulation of apoptosis after CAP treatment in triple negative breast cancer cell line MDAMB-231.

FIGS. 13A-13B are bar graphs showing the differential gene expression of genes involved in the caspase family (FIG. 13A) and caspase activation (FIG. 13B) regulation of apoptosis after CAP treatment in triple negative breast cancer cell line MDA-MB-231.

FIG. 14 is a bar graph showing the differential gene expression of genes involved in the regulation of oxidative stress after CAP treatment in triple negative breast cancer cell line MDA-MB-231.

FIGS. 15A-15C are bar and line graphs showing the differential gene expression of TNF (A) and BCL2A1 (B) transcripts after CAP treatment at 120 power for 5 mins on breast cancer cell line MDA-MB-231. The samples were normalized to 18s rRNA and the fold change were compared to mock controls. Line graph (C) showing the correlation of BCL2A1 and TNF gene expression after CAP treatment at 120 power for 5 mins on two triple negative breast cancer cell line MDA-MB-231 and Hs578T cells. The samples were normalized to 18s rRNA and the fold change were compared to mock controls. Error bars represent means±SEM. Student/test significant at *p values<0.05, *p values<0.01.

FIGS. 16A-16B illustrate expression of BCL2A1 after CAP treatment (CP) and mock controls (M) in triple cancer breast cancer cell line MDA-MB-231. Representative Western blot analysis also revealed that BCL2A1protein is expressed only after cold atmospheric plasma (CAP) treatment in triple negative breast cancer cells MDA-MB-231 cells and mock control cells did not show any detectable protein bands (FIGS. 16A-16B) throughout the incubation period, suggesting that BCL2A1 expression occurs only after induction of stress such as CAP treatment. However, there were no significance in protein expression among 3, 6, 12 and 24 hours groups after CAP treatment.

FIG. 17 is a bar graph showing the effects of BCL2A1 esiRNA silencing on cell viability and the fold change of BCL2A1 mRNA expression in MDA-MB-231 cell line in combination with CAP treatment. At 24 hrs. after transfection with BCL2A1 esiRNA (13 pmol), as mentioned in methods section survival of treatments was determined by MTT assay and BCL2A1 fold change by qRTPCR. The error bars represent means±SEM. (n=3). (****P<0.0001 *p<0.05, **p<0.01, ***p<0.001, Student's t-test) versus control.

FIG. 18A shows representative pictures of IncuCyte live cell image showing the live state of MDA-MB-231-Cell Cycle Green/Red reagent stable cells treated with/without CAP treatment or siRNA BCL2A1. CAP treated cells recover 50% of the times but when transfected with siRNA-BCL2A1 in combination CAP such recover is inhibited.

FIG. 18B shows the line graph for the quantification of the cells in G1, G1-S, S/G2/M, and M-G1 phases after CAP treatment corresponding to section A.

FIGS. 19A-19B show viability of triple cancer breast cancer cell line MDA-MB-231 with Gambogic acid, an antagonist of antiapoptotic Bcl-2 family (FIG. 19A) or CPI203 (FIG. 19B), a BET bromodomain inhibitor after CAP treatment. Bar graph represents MDA-MB-231 MTT cell viability assay after treatment with Gambogic acid (FIG. 19A) or CPI203 (FIG. 19B) in combination of with/without CAP treatment at 80 p Or 120 p 5 mins at 3 L/min He. The samples were compared to the mock control groups. The data is represented by the SEM (n=3). (*p<0.05, **p<0.01, ***p<0.001, Student's t-test).

### DETAILED DESCRIPTION OF THE PREFERRED EMBODIMENTS

Breast cancer is the leading cause of cancer death among women. Predominantly, the poor prognosis is due to the triple-negative breast cancer characterized by the absence or low-level expression of estrogen (ER), progesterone (ER), and HER2 receptors. Cold atmospheric plasma (CAP) delivered to cancer cells induces cell death in triple-negative breast cancer cell line without thermal damage. The present inventors investigated the gene expression profile when CAP is applied by screening the expressions of apoptotic and oxidative stress related gene markers in breast cancer cell lines after CAP treatment to determine the molecular mechanism of CAP induced cell death. Six different types of breast cancer cell lines including MCF-7 and T-47D (luminal A: ER⁺ PR^{+/-}HER2⁻), BT-474 (luminal B: ER⁺PR^{+/-}HER2⁺), SK-BR-3 (ER⁻PR⁻HER2⁺), MDA-MB-231 and Hs578T (basal-like: ER⁻PR⁻HER2⁻) were tested with CAP at two power settings (80 p and 120 p, which are approximately 15 W, and 28 W respectively). Gene expression of 48 apoptotic and 35 oxidative gene markers were determined at four different time points (3 hrs, 6 hrs 12 hrs and 24 hrs) after treatment with CAP, using quantitative real-time polymerase chain reaction (qRT-PCR). After CAP treatment, the expression level of BCL2A1 and TNF were significantly increased in triple-negative cell lines, MDA-MB-231 and Hs578T (p<0.01). In contrast, the HER2-positive and ER, PR positive cell lines showed little or no expression of BCL2A1 (p<0.01). Silencing or inhibiting BCL2A1 mRNA by siRNA increased the potency of the CAP treatment. Combination of CAP and CPI203, a BET bromodomain inhibitor, dramatically increased the CAP-induced cell death without any cytotoxicity by itself (p<0.05). The BCL2A1 expression is induced after CAP treatment, and BCL2A1, acts as potential key gene in breast cancer survival, thereby improving our understanding of molecular profiles of CAP induced cell death. The upregulation of BCL2A1 mRNA levels and expression of protein only after CAP application in the triple-negative subtype could correspond to an important molecular mechanism for their poor prognostic significance which could be reversed with a combination of siRNA or BCL2A1 antagonist-immuno-CAP therapy.

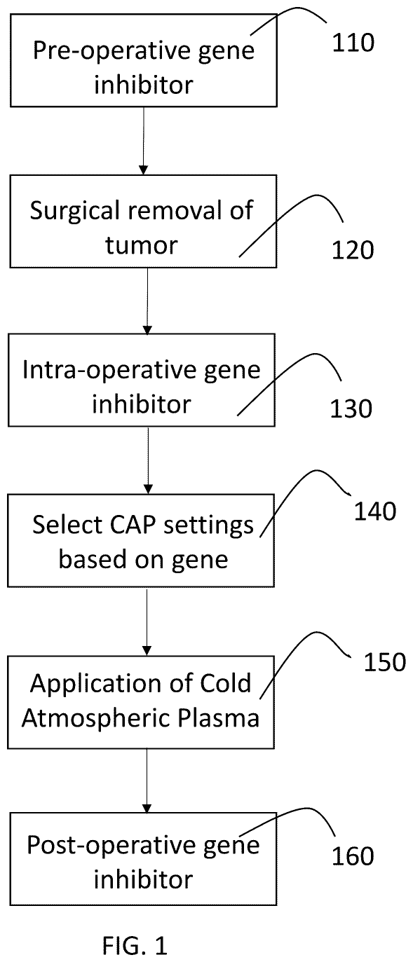

A method for treating breast cancer in accordance with a preferred embodiment of the present invention is shown in FIG. 1. The patient is given a gene inhibitor to inhibit an identified “survival gene” pre-operatively (neoadjuvant) to inhibit the identified gene (110). For triple negative breast cancer, the survival gene is BCL2A1. The pre-operative treatment may be, for example, 24 hours prior to surgery. For triple negative breast cancer, the gene inhibitor may be CPI203, a BET bromodomain inhibitor that inhibits upregulation of BCL2A1. The cancerous tumor is then surgically removed (120). The surgical removal may be via open surgery, robotic surgery, laparoscopic surgery, endoscopic surgery, or any other type of surgery. The gene inhibitor is given to the patient again intra-operatively (130). CAP settings are selected by the CAP system based upon the particular survival gene involved (140). For BCL2A1, the selected setting may be 80 p (15 W). Cold plasma is then applied to the margins of the area from which the cancerous tumor was removed (150). The gene inhibitor is then given to the patient again post-operatively (adjuvant) (160), for example 24 hours after surgery.

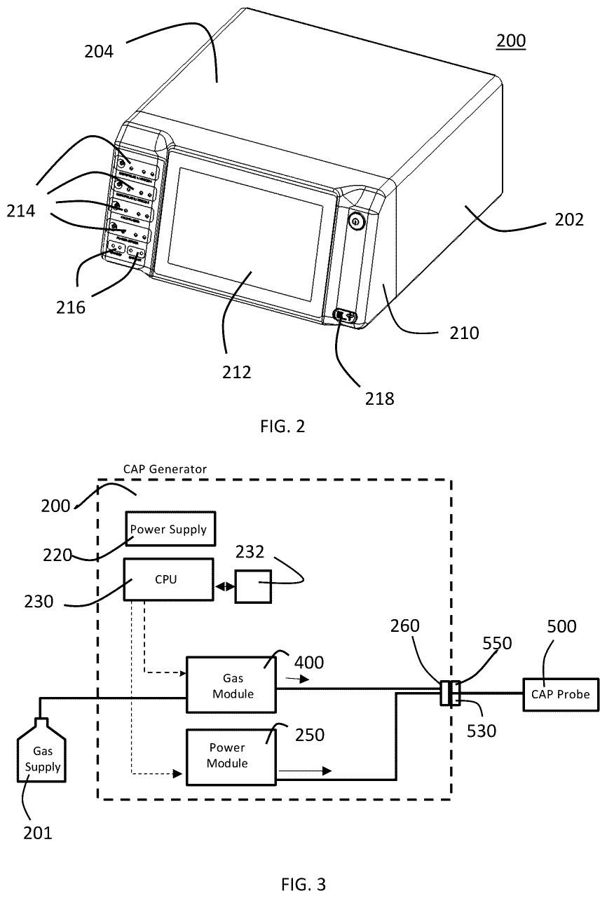

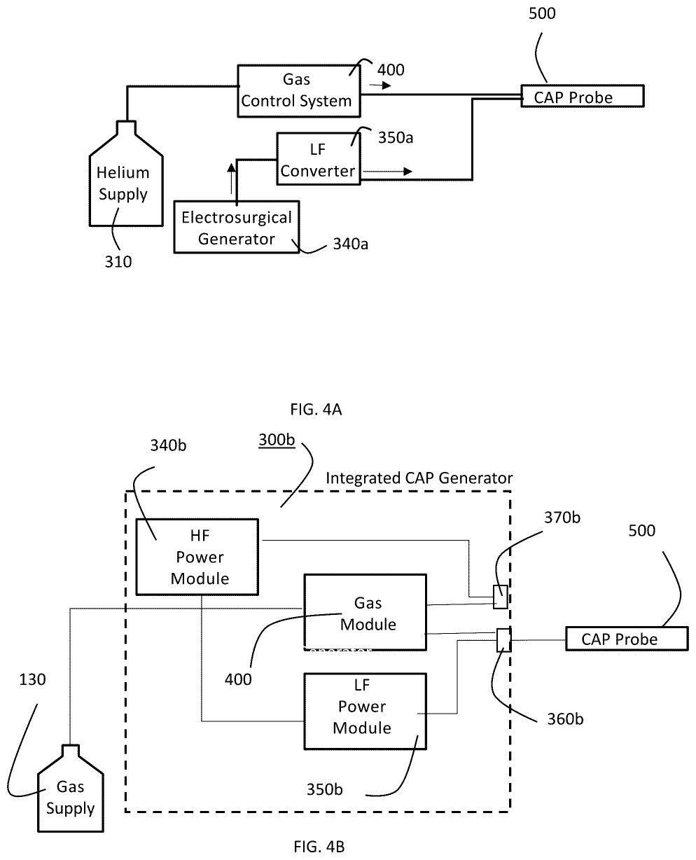

A preferred embodiment of a CAP enabled generator is described with reference to the drawings. A gas-enhanced electrosurgical generator 200 in accordance with a preferred embodiment of the present invention is shown in FIGS. 2 and 3. The gas-enhanced generator has a housing 202 made of a sturdy material such as plastic or metal similar to materials used for housings of conventional electrosurgical generators. The housing 202 has a removable cover 204. The housing 202 and cover 204 have means, such as screws, tongue and groove, or other structure for removably securing the cover to the housing. The cover 204 may comprise just the top of the housing or multiple sides, such as the top, right side and left side, of the housing 202. The housing 202 may have a plurality of feet or legs (not shown) attached to the bottom of the housing. The bottom of the housing 202 may have a plurality of vents (not shown) for venting from the interior of the gas-enhanced generator.

A generator housing front panel 210 is connected to the housing 202. On the face front panel 210 there is a touchscreen display 212 and there may be one or a plurality of connectors 214 for connecting various accessories to the generator 200. For a cold atmospheric plasma generator such as is shown in FIG. 3, for example, there is a connector 260 for connecting a cold atmospheric probe 500. An integrated multi-function electrosurgical generator, such as is shown in FIG. 4B the plurality of connectors may include an argon plasma probe, a hybrid plasma probe, a cold atmospheric plasma probe, or any other electrosurgical attachment. The face of the front panel 210 is at an angle other than 90 degrees with respect to the top and bottom of the housing to provide for easier viewing and use of the touch screen display 212 by a user.

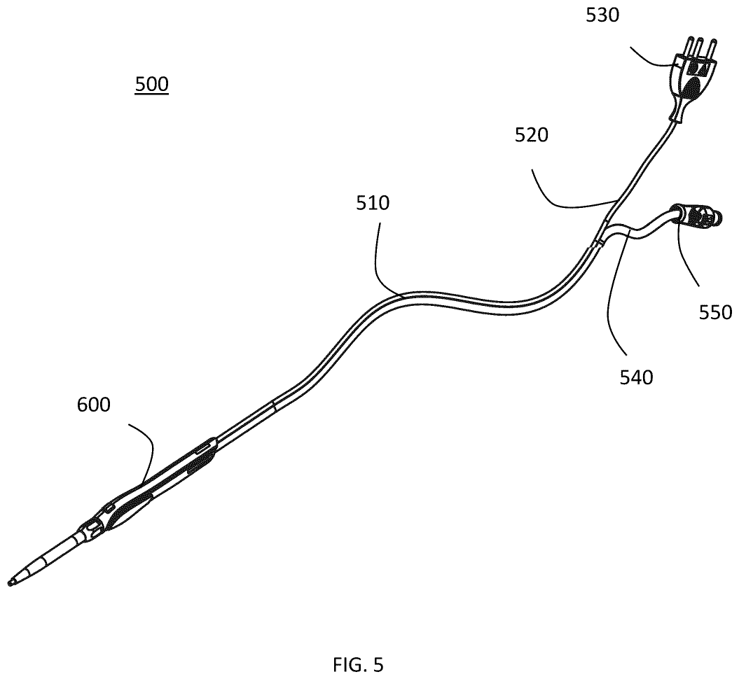

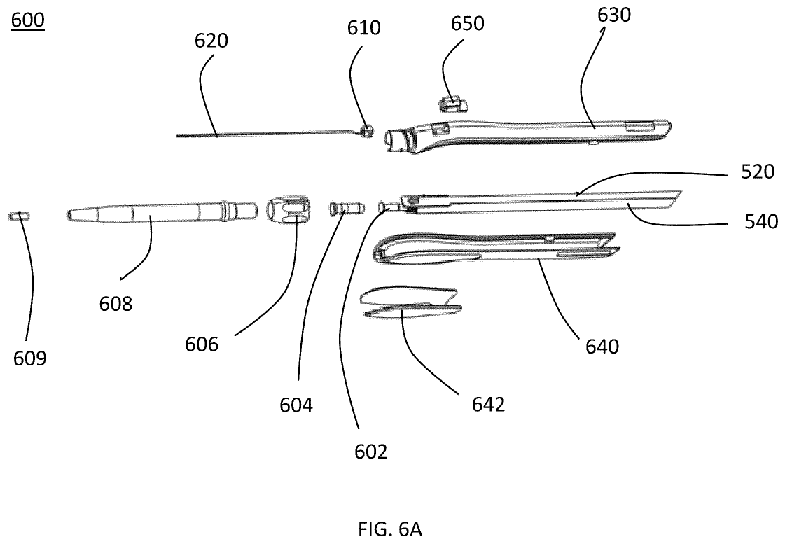

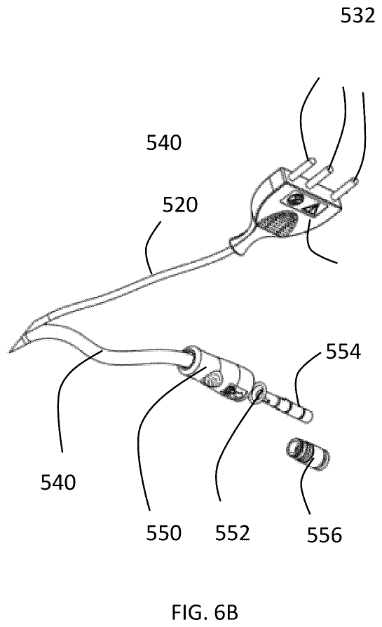

As shown in FIG. 3, an exemplary cold atmospheric plasma (CAP) generator 200 has a power supply 220, a CPU (or processor or FPGA) 230 and a memory or storage 232. The system further has a display 212 (FIG. 2), which may be the display of a tablet computer. The CPU 230 controls the system and receives input from a user through a graphical user interface displayed on display 212. The CAP generator further has a gas control module 400 connected to a source 201 of a CAP carrier gas such as helium. The gas control module 400 may be, for example, of the design described in International Patent Application No. WO 2018/191265, which is hereby incorporated by reference. The CAP generator 200 further has a power module 250 for generating low frequency radio frequency (RF) energy, such as is described in U.S. Pat. No. 9,999,462, which is hereby incorporated by reference in its entirety. The power module 250 contains conventional electronics and/or transformers such as are known to provide RF power in electrosurgical generators. The power module 250 operates with a frequency between 10-200 kHz, which is referred to herein as a “low frequency,” and output peak voltage from 3 kV to 6 kV and preferably at a frequency near (within 20%) of 40 Hz, 100 Hz or 200 Hz. The gas module 400 and power module 250 are connected to connector 260 that allows for attachment of a CAP applicator 500 (as shown in FIGS. 5, 6A and 6B) to be connected to the generator 200 via a connector having an electrical connector 530 and gas connector 550.

As shown in FIG. 4B, other arrangements for delivery of the carrier gas and the electrical energy may be used with the invention. In FIG. 4B, an integrated CAP generator 300b is connected to a source 310 of a carrier gas (helium in this example), which is provided to a gas control system 400, which supplies the gas at a controlled flow rate to CAP applicator 500. A high frequency (HF) power module 340b supplies high frequency (HF) energy to a low frequency power module (converter) 350b, which outputs electrical energy having a frequency in the range of 10 kHz to 200 kHz and an output voltage in the range of 3 kV to 6 Kv. This type of integrated generator will have both a CAP connector 360b for connecting a CAP applicator or other CAP accessory and a connector 370b for attaching HF electrosurgical attachments such as an argon plasma or hybrid plasma probe (not shown).

Another embodiment, shown in FIG. 4A, has a carrier gas source 310 connected to a conventional gas control system 370, which in turn is connected to the CAP applicator 500, and a conventional electrosurgical generator 340 connected to a low frequency (LF) converter 350a, which is then connected to the CAP probe 500.

Other embodiments of cold plasma generation also may be used with the present invention. For example, it may be used with a surgical plasma accessory with distance sensing or a generator with adaptive non-thermal plasma control to produce non-thermal plasma.

In the above-disclosed embodiment, a cold atmospheric plasma below 37° C. is produced. When applied to the tissue surrounding the surgical area, the cold atmospheric plasma induces metabolic suppression in only the tumor cells and enhances the response to the drugs that are injected into the patient.

The cold plasma applicator 500 may be in a form such as is disclosed in U.S. Pat. No. 10,405,913 and shown in FIGS. 5, 6A and 6B. A hand piece assembly 600 has a top side piece 630 and a bottom side piece 640. A control button 650 extends from the interior of the hand piece through an opening in the top side piece 630. Within the hand piece 600 is body connector funnel 602, PCB board 608, electrical wiring 520 and hose tubing (PVC medical grade) 540. The wiring 520 and hose tubing 540 are connected to one another to form a wire and tubing bundle 510. A grip over mold 642 extends over the bottom piece portion 640. In other embodiments, a grip may be attached to the bottom piece 640 in other manners. A probe or scalpel assembly is attached to the end of the hand piece. The probe assembly has non-bendable telescoping tubing 606, a ceramic tip 609, a column nut or collet 606 and body connector tubing 604. The hose tubing 540 extends out of the proximal end of the hand piece to a body gas connector 550, which has an O-ring 552, gas connector core 554 and gas connector tip 556 for connecting to a connector on a gas-enhanced electrosurgical generator. The printed circuit board 608 connects to electrical wiring 520 which leads to electrical connector 530 having electrical pins 532. Inside the handpiece 600 is an electrode 620 and conductive connector 610. There is a control button 650 for controlling the application of electrical energy.

### Experiments

### Materials and Methods

### Cold Plasma Device

A cold plasma system was used for performing all experiments at the Jerome Canady Research Institute for Advanced Biological and Technological Sciences, Takoma Park, MD, USA. The electrosurgical device consists of the USMI SS-601 MCa high-frequency electrosurgical generator (USMI, Takoma Park, MD, USA) integrated with a USMI Canady Cold Plasma Conversion Unit and connected to a Canady Helios Cold Plasma TM Scalpel. The conversion unit has three connectors: a gas connector (to a helium tank), and electrical connector (to the generator), and an electro-gas connector (to the scalpel). The conversion unit also features a high voltage transformer that up-converts voltage up to 4 kV, down-converts frequency to less than 300 kHz, and down-converts power less than 40 W. Additional details and schematics on plasma generation by CCPCS can be found in our previous study. The helium flow rate was set to a constant 3 L/min and the power was set to 80 and 120 P. The plasma scalpel tip was placed 1.5 cm above the surface of the cell media and remained unmoved for the duration of the treatment. The CAP treatment was performed in a laminar flow tissue culture hood, Purifier Logic+ Class II, Type A2 Biosafety Cabinet (Labconco, Kansas City, MO, USA) at room temperature.

### Cell Culture

Human breast cancer cell lines T-47D, SK-BR-3, and BT-474, were purchased from ATCC (Manassas, VA, USA). MCF-7, MDA-MB-231, Hs578T, and HCC1806 were generously donated by Professor Kanaan's laboratory at Howard University. All cell lines except SK-BR-3 were cultured in Roswell Park Memorial Institute (RPMI) 1640 Medium supplemented with 10% fetal bovine serum (Sigma-Aldrich, St. Louis, MO, USA) and 1% Pen Strep (Thermo Fisher Scientific, Waltham, MA, USA) in a 37° C. and 5% CO2 humidified incubator (Thermo Fisher Scientific, Waltham, MA, USA). The exceptions for culture conditions include T-47D, which was additionally supplemented with 0.5 mg/mL insulin. SK-BR-3 was cultured in McCoy's 5A Medium. When cells reached approximately 80% confluence, cells were seeded at a concentration of 105 cells/well into 12-well plates (USA Scientific, Ocala, FL, USA) with a 1 mL media volume per well for cell viability assays.

### Quantitative Real-Time RT-PCR

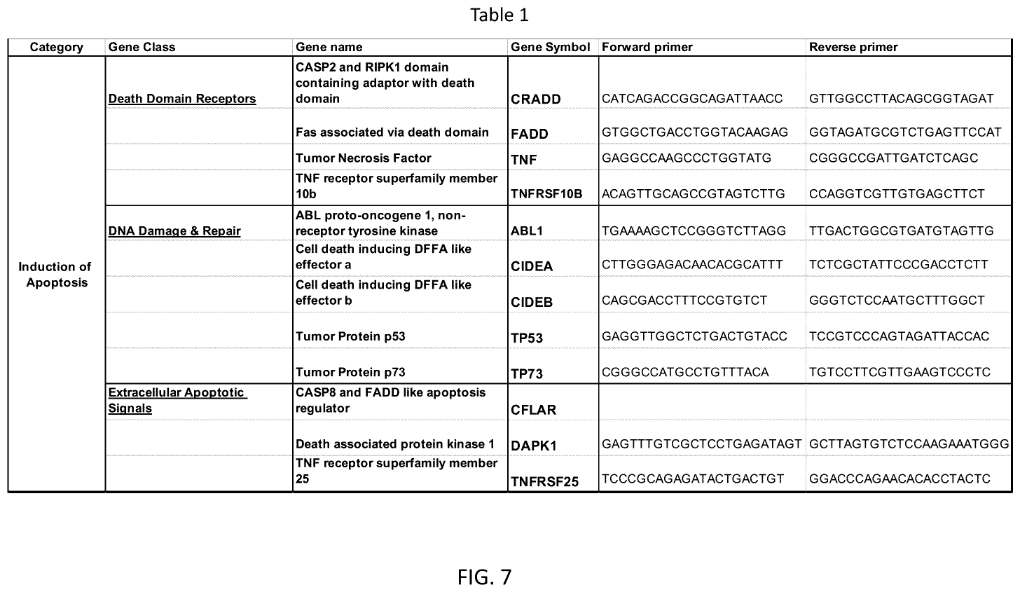

Total RNA was extracted from T-47D, SK-BR-3, BT-474, MCF-7, MDA-MB-231 and Hs578T cell pellets using the TRI reagent and Direct-sol MiniPrep kit (Zymo Research) with DNAse treatment according to the manufacturer's instructions. First-strand cDNA was synthesized with 1 μg of total RNA from these cells using Transcriptor First Strand cDNA Synthesis Kit (Roche Applied Science). Real-time RT-PCR reactions were performed according to the MIQE Guidelines (PMID: 19246619) Quantitative PCR was performed using 1 uL (diluted 1:20 using PCR grade water) of first strand cDNA under the conditions of 95° C. for 15 seconds, annealing at 60° C. for 60 seconds, extension at 72° C. for 30 seconds for 40 cycles, and a final extension at 72° C. for 10 minutes using SYBR Green Master Mix (Applied Biosciences). Primer sequences for analyzing 18S RNA were used for normalization and relative mRNA expression were calculated with 2-ΔΔCT method. Primer sequences for all the 93 genes related to induction of apoptosis, regulation of apoptosis, caspases & regulators and responders of oxidative stress analyzed in this study are listed in Tables 1-4 in FIGS. 7-10.

### Western Blotting

Protein lysates from cell pellets were prepared using RIPA buffer, supplemented with a complete protease inhibitor cocktail (Thermo Fisher Scientific, Waltham, MA) to prevent protein degradation. After centrifugation at 16,000 rcf for 20 min at 4 C, protein concentrations in the supernatants were determined using the Bio-Rad Protein Assay. Twenty grams of protein was denatured at 95° C. for 5 minutes, ran on 4-20% Mini-PROTEAN® TGX Stain-Free™ Protein gels (Bio-Rad), and then transferred onto Trans-Blot Turbo Mini 0.2 μm Nitrocellulose blots (Bio-Rad), according to standard protocols from Bio-Rad Laboratories (Hercules, CA) protocol (PMID: 23709336). After blocking with 5% nonfat milk at 4° C. for one hour, membranes were incubated overnight with gentle agitation at 4° C. in 30 ml of blocking buffer with a mixture containing anti-BCL2A1 a polyclonal antibody from Cell Signaling (Cell Signaling Technology, Inc., Danvers, MA) or Abcam at a 1:50 dilution. After washing the blots were incubated in goat anti-rabbit HRP Ab (Bio-Rad) (1:10000 dilution) in blocking buffer for 1 h with gentle agitation at room temperature. The blots were then incubation in Clarity western ECL substrate chemiluminescent detection reagent (Bio-Rad) for 5 min prior to imaging them on ChemiDoc MP imager (Bio-Rad). Protein band were analyzed by Band Analysis tools of ImageLab software version 4.1 (Bio-Rad) following standard protocol.

Transfection of siRNA

Human BCL2A1 targeting MISSION esiRNA and matching scrambled control esiRNA were purchased from Sigma-Aldrich. Scrambled control esiRNA that does not target any gene was used as the negative control siRNA.

MDA-MB-231 cells were transfected with siRNA and transfection reagent according to the manufacturer's instructions. Transfection of BCL2A1 esiRNA or control esiRNA was done at 13 pmol. Briefly, cells were seeded in a 12-well-plate at a density of 1×10⁵ cells/well overnight in (RPMI) 1640 Medium supplemented with 10% fetal bovine serum (Sigma-Aldrich, St. Louis, MO, USA) and 1% Pen Strep (Thermo Fisher Scientific, Waltham, MA, USA) in a 37° C. and 5% CO2 humidified incubator. The media was replaced with antibiotics-free medium RPMI 2 minutes before the transfection. esiRNA was mixed with Lipofectamine RNAiMAX transfection reagent in 100 μl optimal medium at the required concentration of 13 pmol/mL and were incubated at room temperature for 30 min to form a complex and the mixture was supplemented to each well with optimal medium. Four hours after the transfection, the transfected cells were CAP treated. After 24 hours after the CAP treatment, cells viability was assessed by using MTT assay.

### IncuCyte® Cell Cycle Green/Red Reagent Stable Cell Line Generation.

One day prior to lentiviral transduction, MDA-MB-23 cells were seeded at 1×10⁵ cells/well on 12 well plates (Corning) in media supplemented with 10% fetal bovine serum (Sigma-Aldrich, St. Louis, MO, USA) and 1% Pen Strep (Thermo Fisher Scientific, Waltham, MA, USA) and incubated at 37° C. and 5% CO₂ humidified incubator (Thermo Fisher Scientific, Waltham, MA, USA) for 24 hours. Cell media was changed to fresh antibiotic-free RPMI 1640 with 10% FBS supplemented with 5 μl of 10× virus (˜6 MOI) IncuCyte® Cell Cycle Green/Red Reagent (Sartorius, USA). IncuCyte® Cell Cycle Green/Red Lentivirus Reagent is a fluorescent, single cassette indicator expressing both the GFP (green fluorescent protein) and mKate2 (red fluorescent protein) to distinguish between cells in the G1 and S/G2/M cell cycle phase without altering cell function. The cells were incubated at 37° C. with 5% CO₂ for 24 h. Cell media was changed to fresh RPMI media supplemented with 10% fetal bovine serum, 1% Pen Strep and 5 ug/mL puromycin and incubated for 24 hrs. Cell with strong fluorescence and growth were selected for expansion. Selected cells were maintained in RPMI media supplemented with 10% fetal bovine serum, 1% Pen Strep and 2.5 ug/mL puromycin for all the IncuCyte experiments.

### IncuCyte Live Cell Imaging and Cell Cycle

MDA-MB-231-Cell Cycle Green/Red reagent stable cells were seeded in 12-well plates at a density of 105 cells/well and treated with CAP. The treated cells were then incubated at 37° C. with 5% CO2 and monitored on the IncuCyte® Live Cell Analysis System (Sartorius, USA). The cells were scanned with phase contrast and green channels at 10× magnification at an interval of 1 h for 3 days. Cells were scanned every hour with phase contrast, green, and red channels at 10× magnification with Cell-By-Cell Module for MCF-7 and MDA-MBA-231 cells. For BT-474 cells, which tend to grow in clusters, Cell by Cell Module could not properly detect the boundaries between each cell, basic scanning and analysis was used. After scanning, fluorescent object were quantified using the IncuCyte integrated analysis software with background subtraction.

### Compounds

Gambogic acid and CPI203 were purchased from Med Chem express, USA and Sigma-Aldrich (St Louis, MO, USA) respectively.

### Gambogic Acid and CPI203 Treatment:

Drugs were dissolved in DMSO to make stock solutions and diluted in complete growth media and subsequently added in a triplicate to the wells of 12-well plates.

### Cell Viability Assay

Thiazolyl blue tetrazolium bromide (MTT) assay was performed on the cells 24 hours after plasma treatment following the manufacturer's protocol with all MTT assay reagents purchased from Sigma-Aldrich (St. Louis, MO, USA). The absorbance of the dissolved compound was measured by BioTek Synergy HTX (Winooski, VT, USA) microplate reader at 570 nm following standard procedure.

### Statistics

All viability assays were repeated 3 times with at least 2 replicates each. Data was plotted by Microsoft Excel 2016 as the mean±standard error of the mean. A student t-test or a one-way analysis of variance (ANOVA) was used to check statistical significance where applicable. The differences were considered statistically significant for *p≤0.05. A one-way multivariate analysis of variance (MANOVA) followed by a Post-Hoc test was used to check statistical significance where applicable.

### Results and Discussion

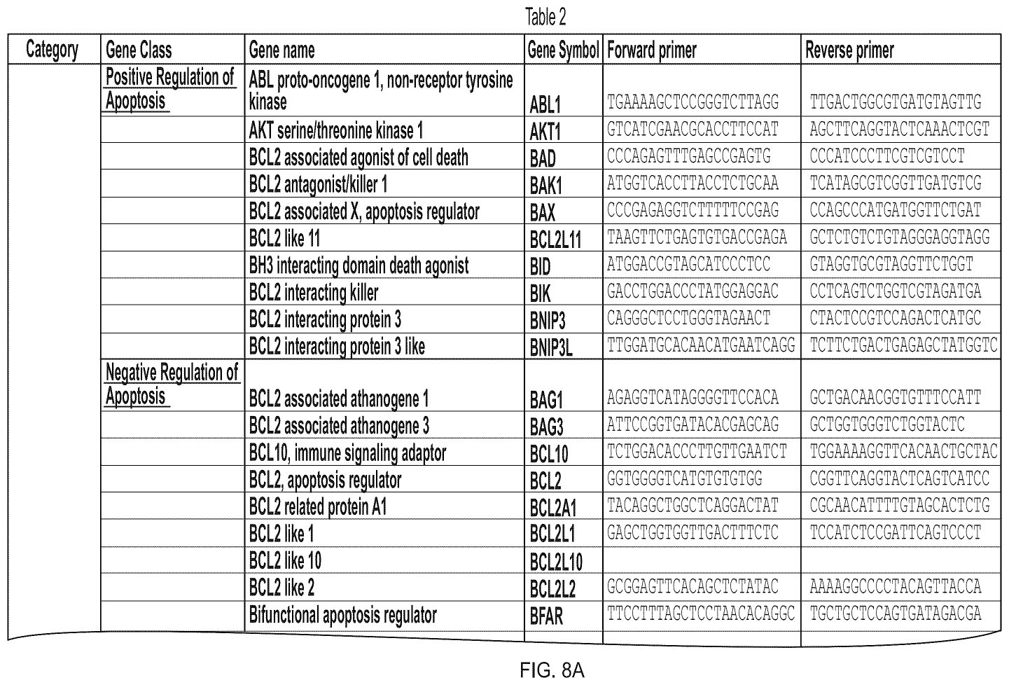

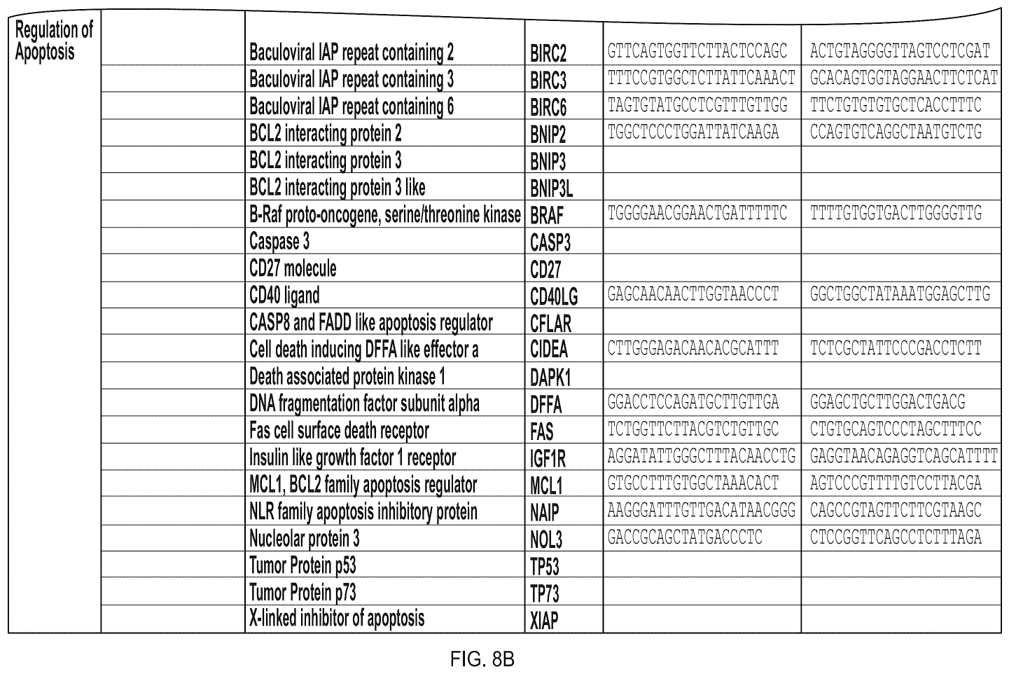

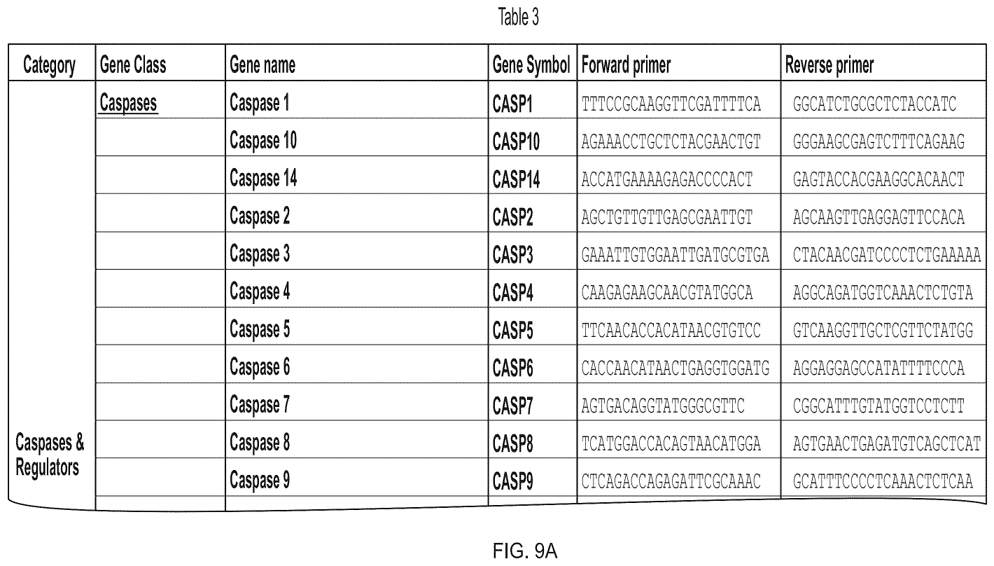

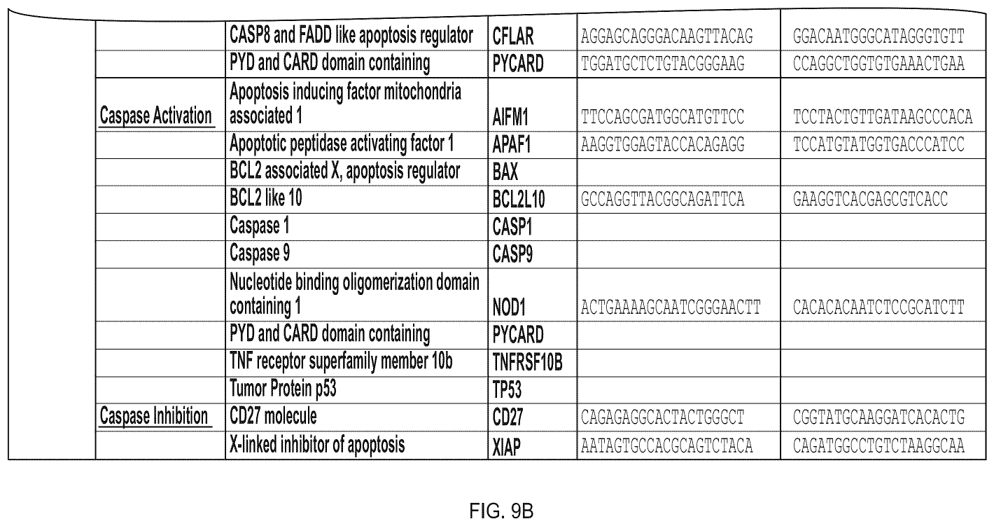

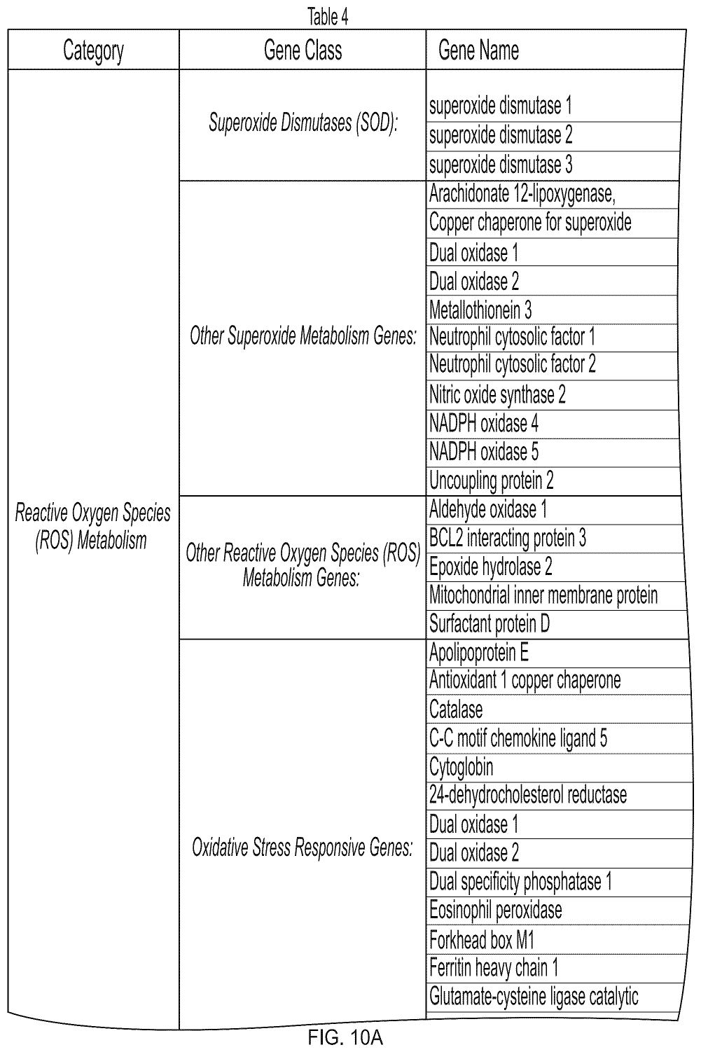

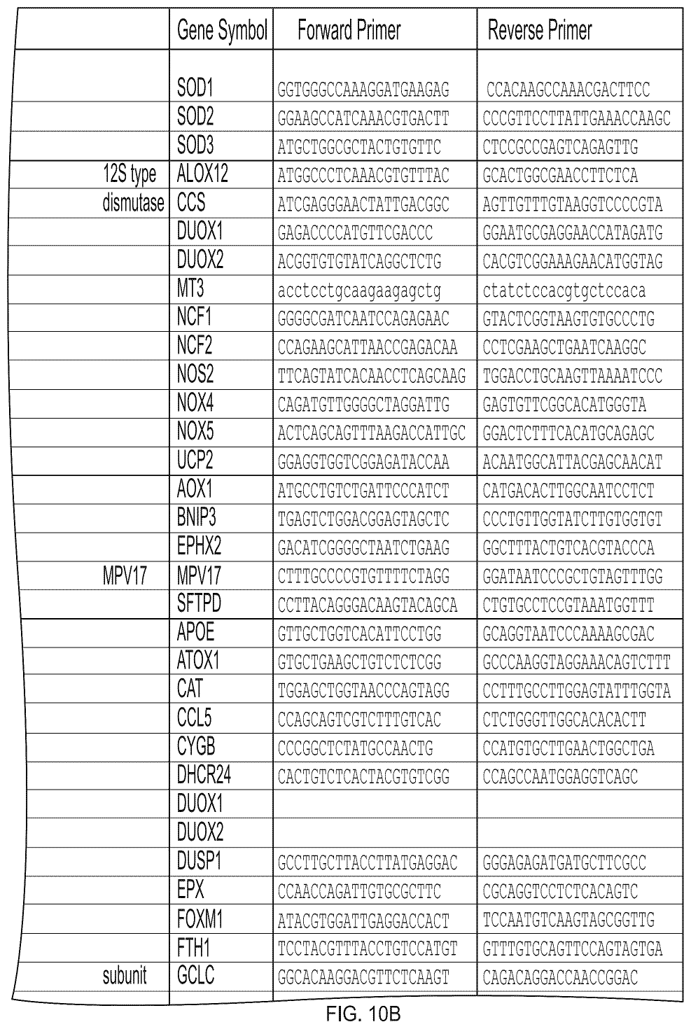

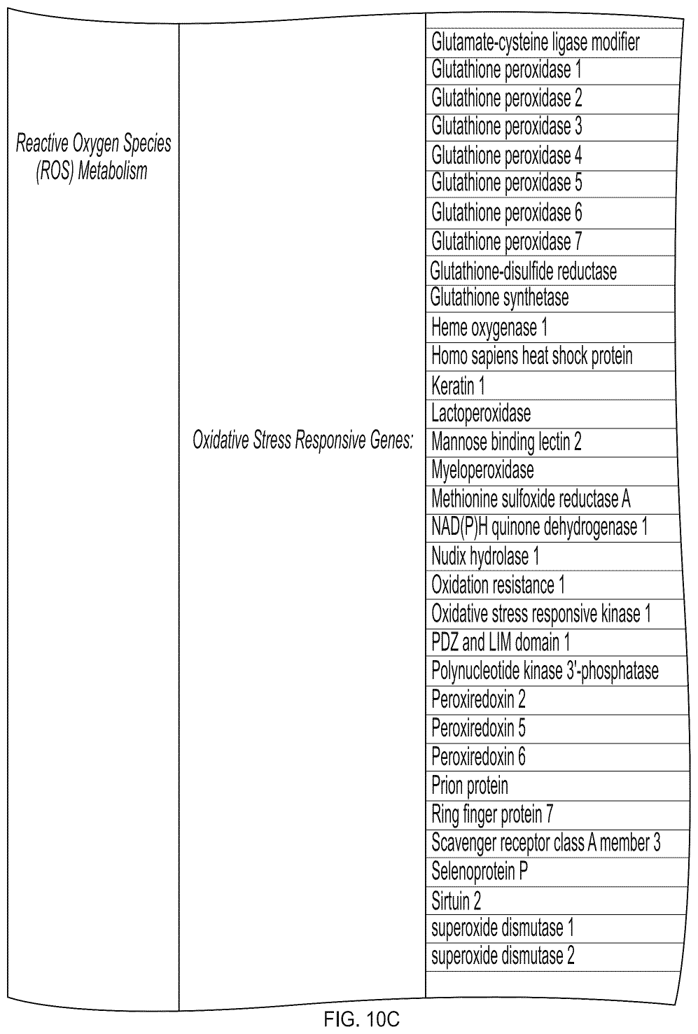

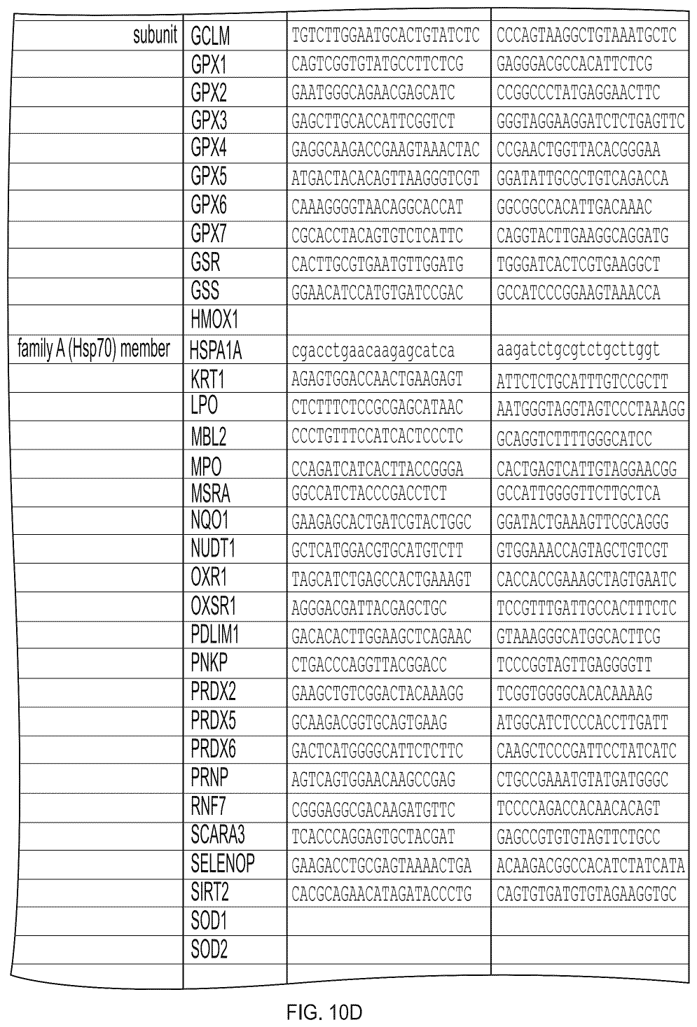

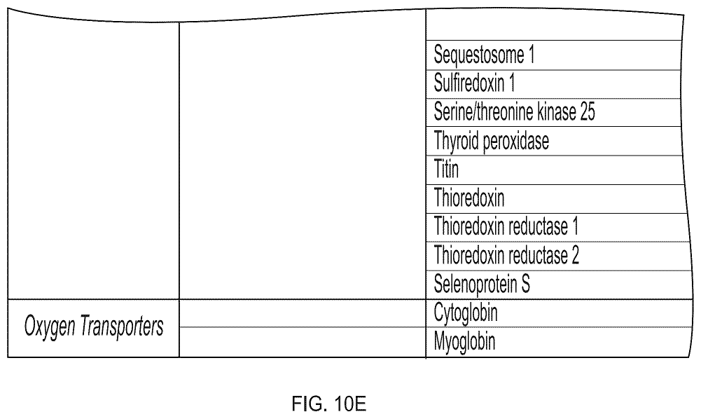

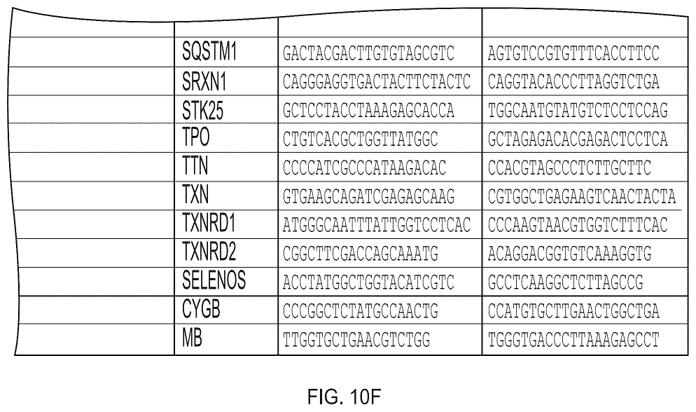

To systematically investigate the molecular basis for survival after CAP treatment by breast cancer cell lines which are classified into their intrinsic subtypes such as luminal A (ER+PR+/−HER2−), luminal B (ER+PR+/−HER2+), basal-like (ER−PR−HER2−), and HER2-positive (ER−PR−HER2+), we conducted quantitative real time PCR analysis to screen the genes that are differentially expressed after CAP treatment. We selected genes from four major categories based on their ability in “induction of apoptosis” (FIG. 7: Table 1), “regulation of apoptosis” (FIGS. 8A-8B: Table 2), “caspases & regulators” (FIGS. 9A-9B: Table 3) and “responders of oxidative stress” (FIGS. 10A-10E: Table 4).

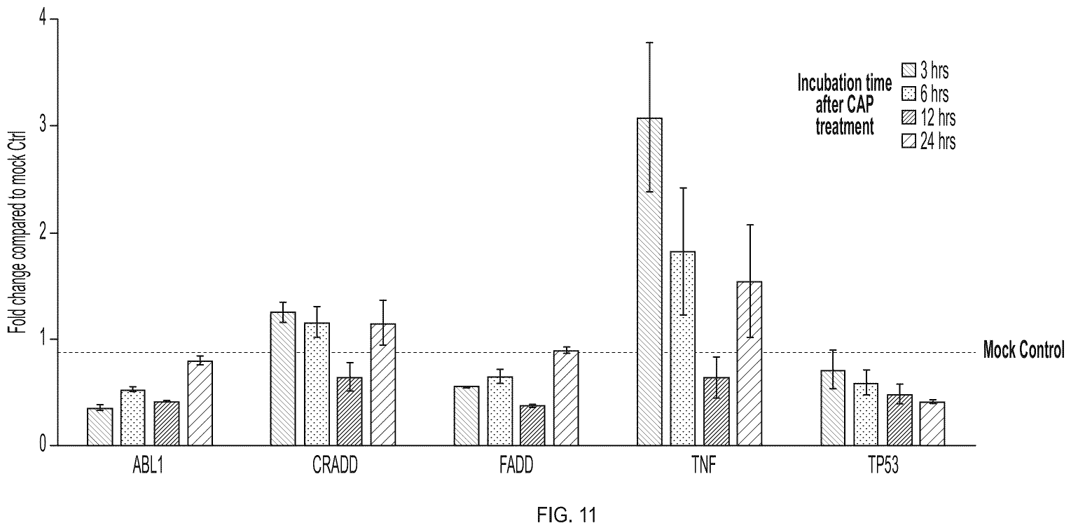

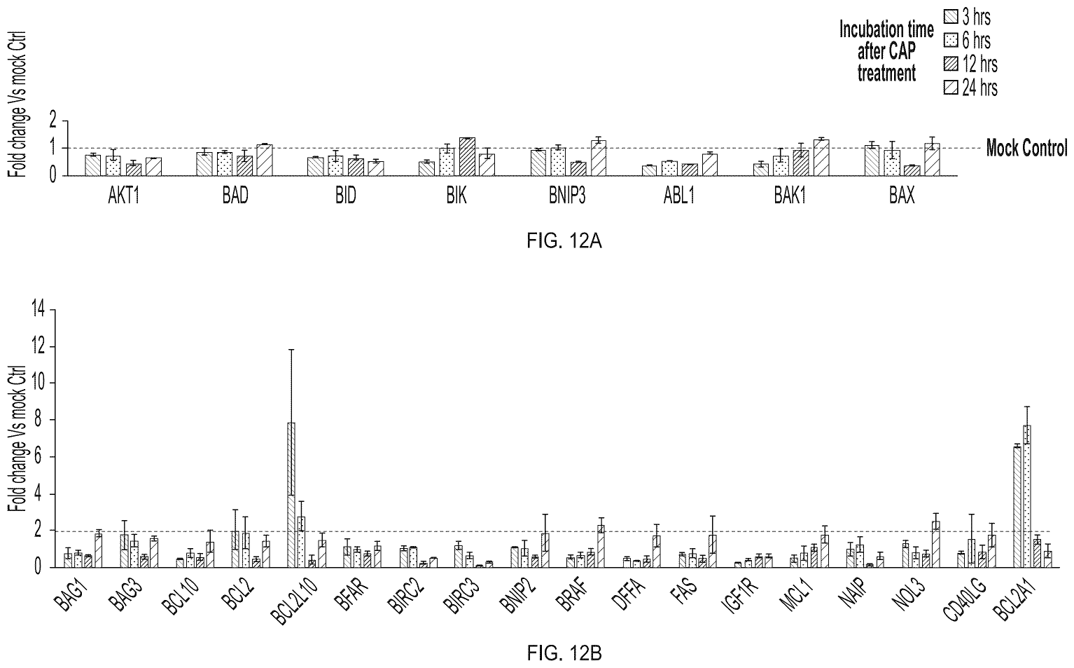

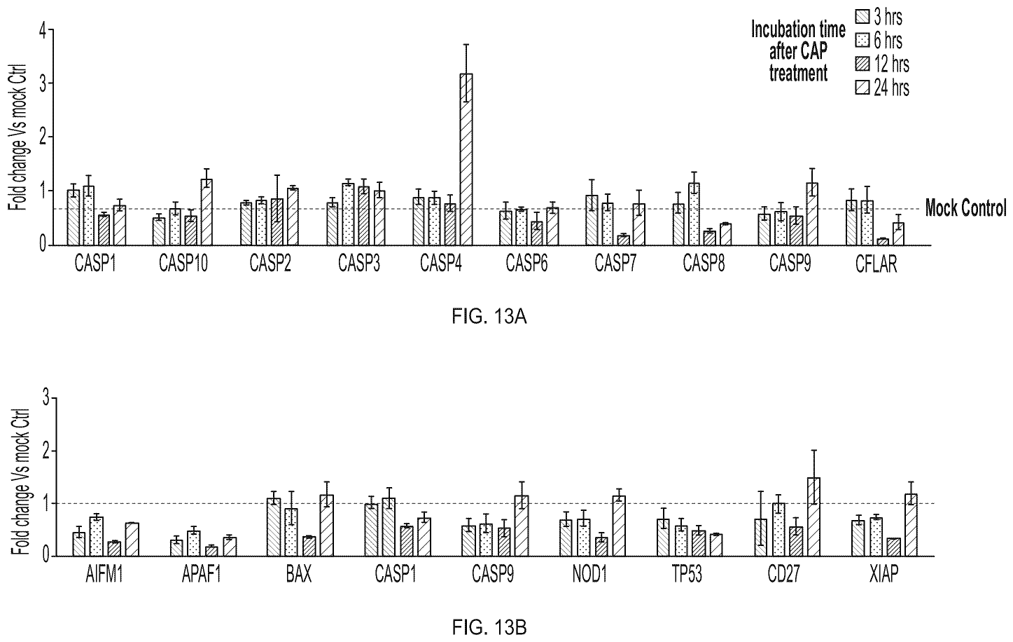

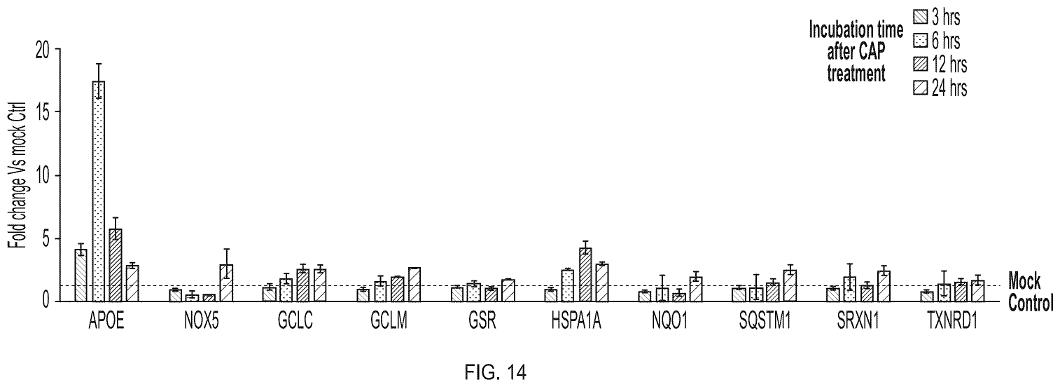

For the purpose of screening, we CAP treated triple negative breast cancer cell line MDA-MB-231 cells at 80 power and 120 power for 5 mins and incubated at 37 C until isolating RNA at 3-, 6-, 12-, and 24-hour time points. Q RT-PCR were carried out for genes involved in the induction of apoptosis (FIG. 11), regulation of apoptosis (FIG. 12), caspase activation (FIG. 13) and regulation of oxidative stress (FIG. 14).

FIG. 11 is a bar graph showing the differential gene expression of genes involved in the induction of apoptosis after CAP treatment in triple negative breast cancer cell line MDA-MB-231. The samples were normalized to 18s rRNA and the fold change were compared to mock controls. Error bars represent means±SEM. Student/test significant at *p values<0.05, **p values<0.01.

ABL proto-oncogene 1, non-receptor tyrosine kinase (ABL1), Fas associated via death domain (FADD) and Tumor Protein p53 (TP53) mRNA expression were significantly (p<0.05) down regulated after CAP treatment compared to untreated mock samples, whereas death associated protein kinase 1 (DAPK1) expression were not detected in either conditions. Tumor Necrosis Factor (TNF) was significantly up-regulated after 3 hrs. of treatment (p<0.01). Although not statistically significant 6 hrs. and 24 hrs. incubation time point showed upward trend in expression suggesting TNF is involved in the induction of apoptosis in CAP treated breast cancer cells (FIG. 11).

Among genes involved in apoptosis regulation, B-cell lymphoma 2-related protein A1 (BCL2A1)/Bcl-2-related gene expressed in fetal liver (Bfl-1) and BCL2 like 10 (BCL2L10) mRNA transcripts showed significant up regulation (p<0.001 and p<0.05) (FIG. 2B). BCL2A1 mRNA was at least 4 folds higher at 3 hours and 6 hours incubation time point after CAP treatment and its expression is above baseline only after CAP treatment. AKT serine/threonine kinase 1 (AKT1) mRNA expression is down regulated throughout the 24 hrs. time point incubation after CAP treatment breast cancer cells (p<0.05). BCL2 associated agonist of cell death (BAD) mRNA expression is relatively unchanged. Whereas, BH3 interacting domain death agonist (BID) mRNA expression is down regulated throughout the 24 hrs. time point (p<0.05). BCL-2 Interaction Killer (BIK) mRNA expression is down regulated at 3 hrs. time point (p<0.05) but steadily recovered above the baseline expression by the time point of 12 hrs. and 24 hours incubation. BCL2 interacting protein 3 (BNIP3) mRNA expression is relatively unchanged at 3 and 6 hrs incubation after CAP treatment but is down regulated at 12 hrs and recovered above the baseline expression at the 24 hrs time point. BCL2 associated X, apoptosis regulator (BAX) mRNA expression is remains at baseline levels at 3, 6 and 24 hrs. incubation time point but is significantly down regulated at 12 hrs. (p<0.05). B-cell lymphoma 2 (BCL2) and BCL2 associated athanogene 3 (BAG3) mRNAs were up regulated at 3 and 6 hrs incubation time point and down regulated by 12 hrs. time point and again up regulated at 24 hrs. incubation time but this differentially regulation in response to the CAP treatment was not statistically significant. BCL2 associated athanogene 1 (BAG1) and BCL2 like 10 (BCL2L10), Bifunctional apoptosis regulator (BFAR), BCL2 family apoptosis regulator (MCL1), Nucleolar protein 3 (NOL3) mRNA expressions follow a similar trend by remaining at baseline levels at 3 and 6 hrs. incubation time point but is slightly down regulated at 12 hrs and up regulated at 24 hrs. incubation time point. Even though differentially regulated during the later stage of the apoptosis process initiated by the CAP treatment they are not statistically significant (FIG. 3B). Expect for CASP4 all the other Caspases that we analyzed were either unchanged or down regulated after CAP treatment (FIG. 3A). CASP4 mRNA was up regulated at least ˜4 folds at 24 hrs. incubation time point after CAP treatment (p<0.01).

FIGS. 12A-12B are bar graphs showing the differential gene expression of genes involved in the positive (FIG. 12A) and negative (FIG. 12B) regulation of apoptosis after CAP treatment in triple negative breast cancer cell line MDA-MB-231. The samples were normalized to 18s rRNA and the fold changes were compared to mock controls. Error bars represent means±SEM. Student/test significant at *p values<0.05, **p values<0.01.

FIGS. 13A-13B are bar graphs showing the differential gene expression of genes involved in the caspase family (FIG. 13A) and caspase activation (FIG. 13B) regulation of apoptosis after CAP treatment in triple negative breast cancer cell line MDA-MB-231. The samples were normalized to 18s rRNA and the fold change were compared to mock controls. Error bars represent means±SEM. Student/test significant at *p values<0.05, *p values<0.01.

Caspase 1 (CASP1), Caspase-1/Interleukin-1 converting enzyme (ICE) can induce pyroptosis, a lytic form of cell death and also been shown to induce necrosis. Caspase 2, 4, 5, 7 and 8 are involved in the cascade of caspases responsible for apoptosis execution. Caspase 3 (CASP3) protein interacts with caspase-8 and caspase-9 and is activated in the apoptotic cell both by extrinsic (death ligand) and intrinsic (mitochondrial) pathways. Caspase 6 (CASP6) protein is processed by caspases 7, 8 and 10, and is thought to function as a downstream enzyme in the caspase activation cascade. Expect for CASP4 all the other Caspases that we analyzed were either unchanged or down regulated after CAP treatment.

FIG. 14 is a bar graph showing the differential gene expression of genes involved in the regulation of oxidative stress after CAP treatment in triple negative breast cancer cell line MDA-MB-231. The samples were normalized to 18s rRNA and the fold change were compared to mock controls. Error bars represent means±SEM. Student/test significant at *p values<0.05, *p values<0.01.

Among all the genes involved in oxidative stress (NOX5, GCLC, GCLM, GSR, NQO1, SQSTM1 and TXNRD1) Apolipoprotein E (APOE) was the only genes which up regulated significantly (p<0.001) after CAP treatment at 3-, 6-, 12-, and 24-hours incubation time point.

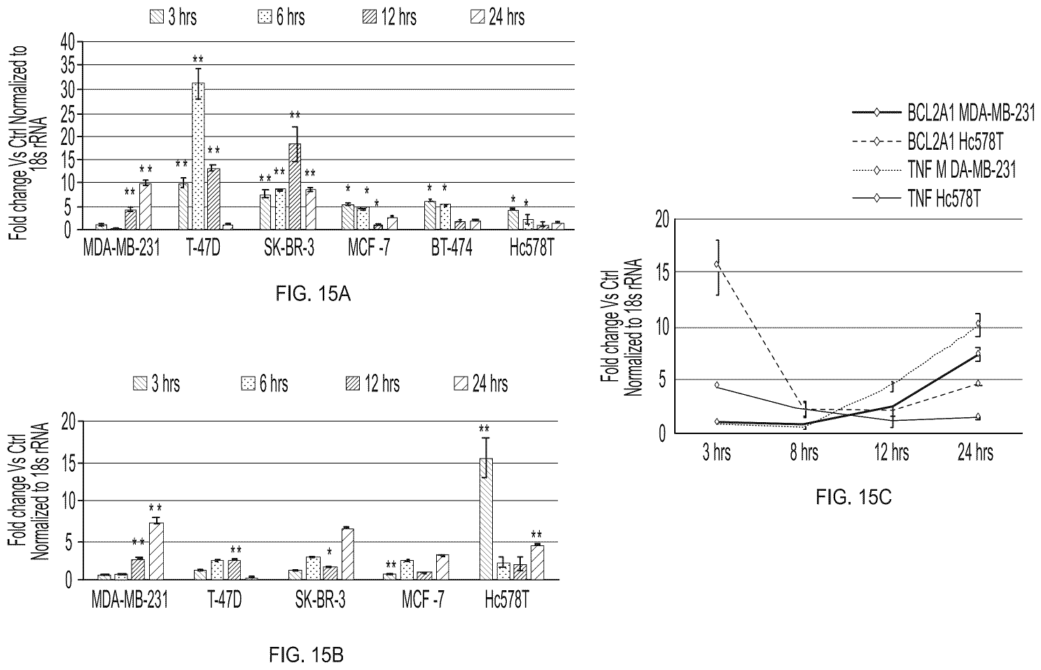

FIGS. 15A-15C are bar and line graph showing the differential gene expression of TNF (A) and BCL2A1 (B) transcripts after CAP treatment at 120 power for 5 mins on breast cancer cell line MDA-MB-231. The samples were normalized to 18s rRNA and the fold change were compared to mock controls. Line graph (C) showing the correlation of BCL2A1 and TNF gene expression after CAP treatment at 120 power for 5 mins on two triple negative breast cancer cell line MDA-MB-231 and Hs578T cells. The samples were normalized to 18s rRNA and the fold change were compared to mock controls. Error bars represent means±SEM. Student/test significant at *p values<0.05, *p values<0.01.

Based on the initially gene profile screen after CAP treatment in triple negative breast cancer cells MDA-MB-231 cells three genes were further analyzed in other breast cancer cell lines MCF-7 (ER+, PR+, HER2−), T-47D 7 (ER+, PR+, HER2−), SK-BR-3 (ER−, PR−, HER2+), BT-474 (ER+, PR+, HER2+), MDA-MB-231 (ER−, PR−, HER2−) and Hs574T (ER−, PR−, HER2−) (FIG. 5). Triple negative cell line Hs574T showed significant up regulation of BCL2A1 in at 3 hrs. after CAP treatment and remain above 2 fold increase up to 24 hours. T-47D 7, SK-BR-3 and BT-474 had very low detection levels of BCL2A1 expression. MCF-7 showed significant 2 fold up regulation of BCL2A1 at 6 and 24 hours' time points. TNF was significantly up regulated in all the cell lines at different time points for incubation after CAP treatment. The trend of BCL2A1 and TNF expressions were correlated in MDA-MB-231 and Hs574T cells suggesting a synergy in their regulation (FIG. 15C).

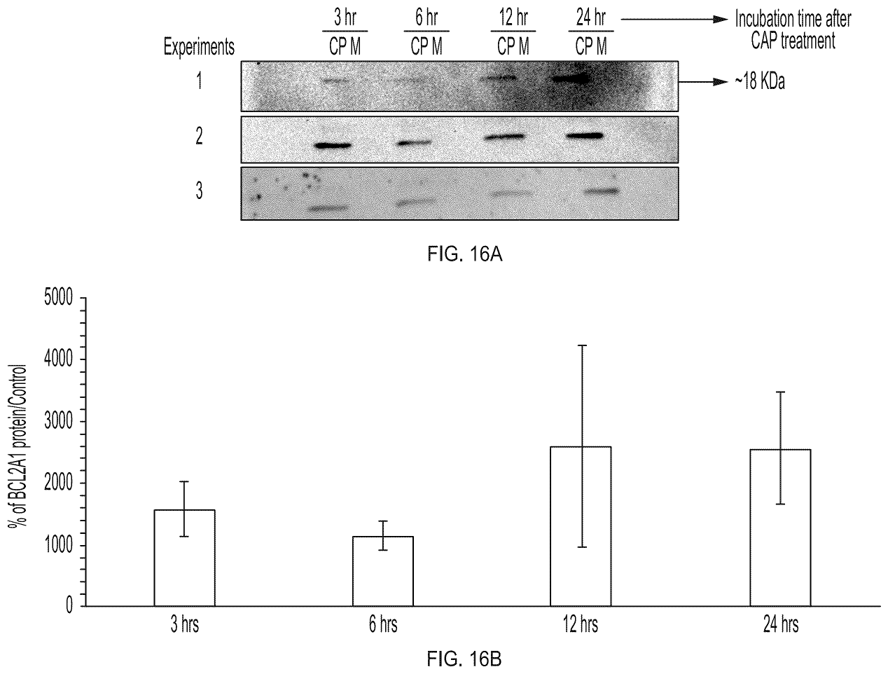

FIGS. 16A-16B illustrate expression of BCL2A1 after CAP treatment (CP) and mock controls (M) in triple cancer breast cancer cell line MDA-MB-231. Representative Western blots (A) and bar graph representation (B) of the quantification of BCL2A1 protein in total tissue lysates from MDA-MB-231 cells after CAP treatment compared to the mock control groups.

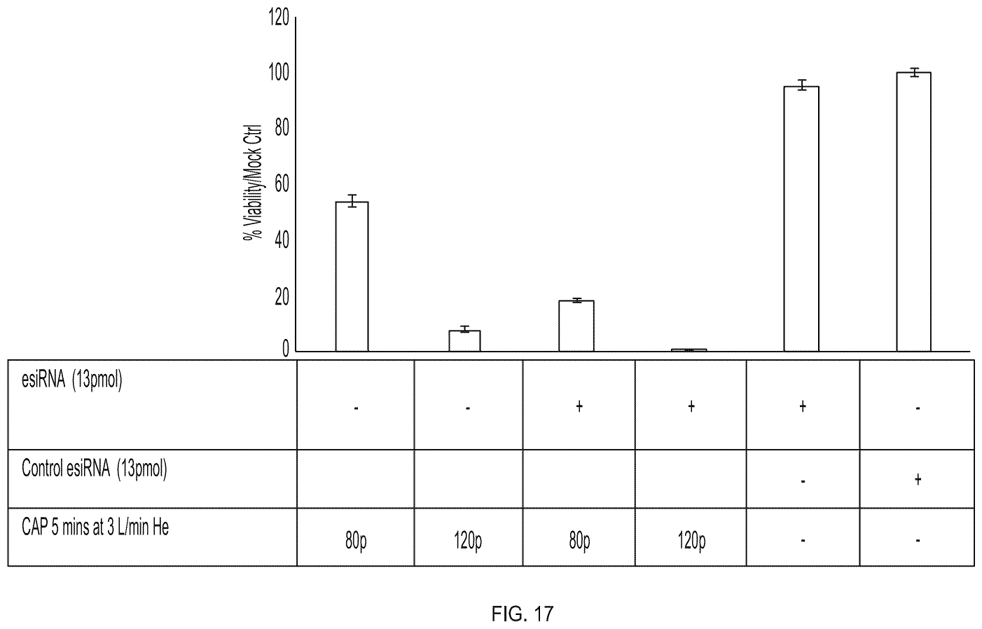

FIG. 17 is a bar graph showing the effects of BCL2A1 esiRNA silencing on cell viability and the fold change of BCL2A1 mRNA expression in MDA-MB-231 cell line in combination with CAP treatment. At 24 hrs. after transfection with BCL2A1 esiRNA (13 pmol), as mentioned in methods section survival of treatments was determined by MTT assay and BCL2A1 fold change by qRTPCR. The error bars represent means±SEM. (n=3). (****P<0.0001 *p<0.05, **p<0.01, ***p<0.001, Student's t-test) versus control.

Specific downregulation (˜5 fold) of BCL2A1 mRNA expression by esiRNAs silencing in MDA-MB-231 cells were carried out to determine the effects of BCL2A1 with and without CAP treatment (FIG. 17) and were analyzed by qRTPCR. The viability of cells were determined by MTT assay (FIG. 17), esiRNA-BCL2A1 has no effect on the MDA-MB-231 cells, but the in combination with CAP treatment at 80 p 5 mins the viability of cells significantly (p<0.001) reduced after 24 hours of treatment compared to either of the treatments alone (FIG. 17).

For live cell image analysis, stables of MDA-MB-231 cells expressing Cell Cycle Green/Red tags were created. The Incucyte Cell Cycle Green/Red tag is a fluorescent, single cassette indicator expressing both the GFP (green fluorescent protein) and mKate2 (red fluorescent protein) to distinguish between cells in the G1 and S/G2/M cell cycle phase without altering cell function. The Incucyte live cell imaging experimental setup is detailed in the method section. MDA-MD-231 stable cells were monitored for cell confluence and G1 (Red) or S/G2/M (Green) or S to G1 transition (S-G1, Yellow) phases of cell cycle for up to 3 days by Incucyte live imaging. The cells treatment with esiRNA-BCL2A1 did not show any changes in cell confluence or cell cycle phases compared to mock cells or esiRNA-control treated cells, but when treated with CAP at 80 p 5 mins the viability of esiRNA-BCL2A1 treated cells were close to 0% by 12 hours and remained the same throughout the incubation period, whereas in CAP only treated group showed 30% viability at 12 hours and recovered to 50% and 70% viability by 24 and 48 hours incubation time points respectively (FIG. 8). The reduction in number of cells at the G1 phase was very pronounced in stable MDA-MB-231 cells with esiRNA-BCL2A1-CAP treatment compared to CAP treatment alone. The number of transitioning from G1 phase to S/G2/M phases were close to 0% in esiRNA-BCL2A1-CAP treated cells whereas CAP treatment alone group showed limited number of cells progressed towards S/G2/M phases (yellow and green) compared control groups (FIG. 8B). After CAP treatment at 80 p 5 mins, the MDA-MB-231 cells at G1 phase are drastically reduced compared to control groups. There is an increase in cells at G1-S phase during initial time points but later the numbers are reduced. The cells at S/G/M phase increase and reach a plateau stage by 12 hrs. All this change is more pronounced in cells treated with siRNA-BCL2A1 in combination with the CAP treatment. In the siRNA-BCL2A1-CAP treated group all most all of the cells are dead by 24 hrs. at S/G/M phase but because the fluorescent proteins are oxidized the fluorescence are retained with incomplete protein degradation. Even though there is no detectable BCL2A1 protein at the baseline level in MDA-MB-231 cells, siRNA-BCL2A1 without the CAP treatment also showed a small increase in the S/G/M phase cells compared to siRNA control and mock groups.

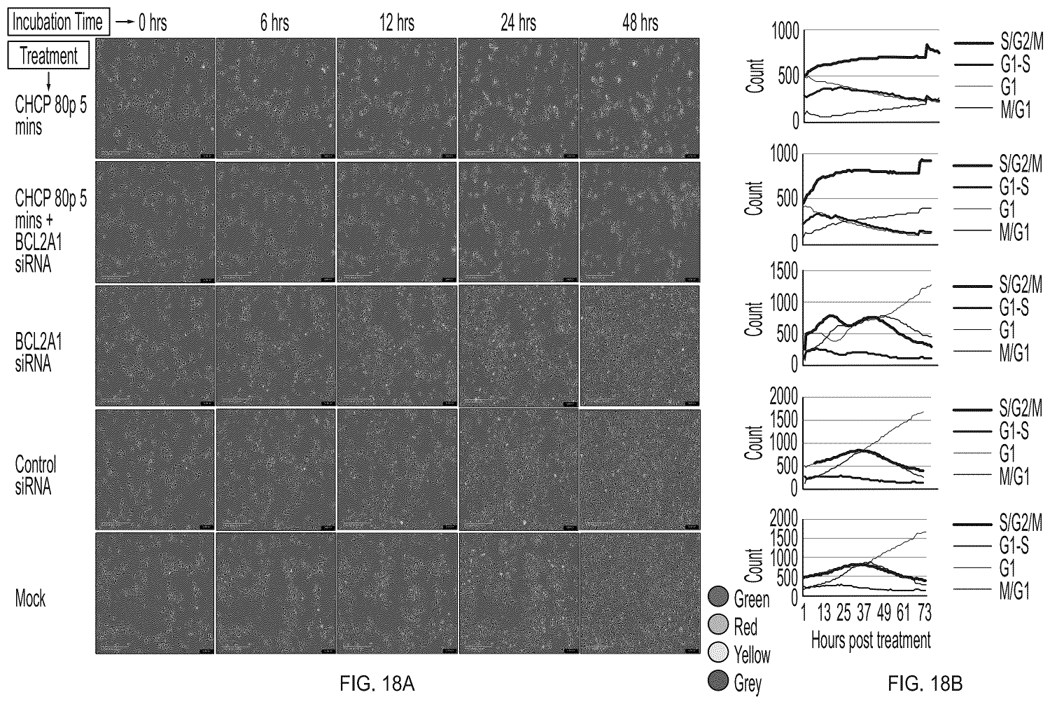

FIG. 18A shows representative pictures of IncuCyte live cell image showing the live state of MDA-MB-231-Cell Cycle Green/Red reagent stable cells treated with/without CAP treatment or siRNA BCL2A1. CAP treated cells recover 50% of the times but when transfected with siRNA-BCL2A1 in combination CAP such recover is inhibited.

FIG. 18B shows the line graph for the quantification of the cells in G1, G1-S, S/G2/M, and M-G1 phases after CAP treatment corresponding to section A.

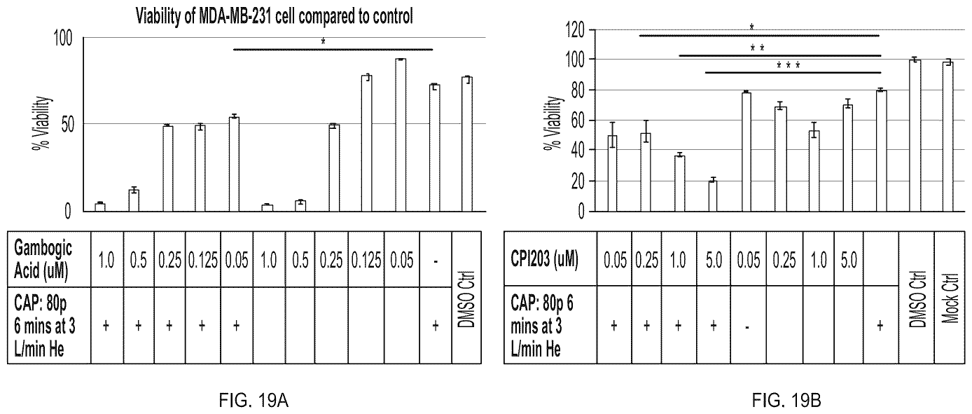

FIGS. 19A-19B show viability of triple cancer breast cancer cell line MDA-MB-231 with Gambogic acid, an antagonist of antiapoptotic Bcl-2 family (FIG. 19A) or CPI203 (FIG. 19B), a BET bromodomain inhibitor after CAP treatment. The bar graph represents MDA-MB-231 MTT cell viability assay after treatment with Gambogic acid (FIG. 19A) or CPI203 (FIG. 19B) in combination of with/without CAP treatment at 80 p Or 120 p 5 mins at 3 L/min He. The samples were compared to the mock control groups. The data is represented by the SEM (n=3). (*p<0.05, **p<0.01, ***p<0.001, Student's t-test).

To determine the if gambogic acid (GA), an antagonist of antiapoptotic Bcl-2 family or CPI203, a BET bromodomain inhibitor that is previously shown to down regulate BCL2A1 would induce increased cell death after CAP treatment by inhibiting BCL2A1 protein (FIG. 9). Baseline toxicity measurements were established for GA and CPI203. GA showed cell viability decreased in all the 5 doses test but there was no dose that significantly reduced the viability of cells in combination with CAP treatment compared to CAP treatment alone. CPI203 showed significant reduction in cell viability at three dose concentrations (0.25 uM; *p<0.05), (1 uM; **p<0.01) and (5 uM; ***p<0.001).

CAP as a promising anti-cancer treatment has been demonstrated in several cancers including TNBC. Previous studies have shown that molecular features and corresponding tumor subtypes of each breast tumor cell lines are feasible as models for tumors of the same subtypes. In this study we are the first to demonstrate gene expression profile screening for 6 different breast cancer cell line with different molecular subtype and show that BCL2A1 is only expressed after treatment in TNBC and silencing of BCL2A1 by siRNA or inhibit its expression by CPI203 increased the potency of CAP treatment. This study also shows that narrowing down and analyzing few particular categories of genes would be very helpful to pinpoint low transcript number gene expression genes that would express only in certain conditions such as CAP treatment. Here, by analyzing four different categories of genes such as induction of apoptosis, regulation of apoptosis, caspases & regulators and reactive oxygen species (ROS) and metabolism specific expression of low transcript number gene such as BCL2A1 gene expression were detected after CAP treatment. This would have not been possible if a high through-put gene expression study were carried out. In fact, Western blot analysis revealed that detectable BCL2A1 protein was only expressed after CAP treatment (FIG. 6).

BCL2A1 protein contains 4 BH-domains and does not have any C-terminal transmembrane domain like other BCL2 family of protein but regulates anti-apoptotic function. BCL2A1 mRNA expression has been shown to be general overexpressed in many cancers including breast cancer. Studies have shown that CD40 signaling, PI3K and ERK signaling initiated by ICAM-1 binding have been found to induce NF-kB and subsequently BCL2A1 expression. With preliminary experimental results we did not see any increase in NF-kB (preliminary results not shown). Nutlin-3, a cis-imidazoline analog and p53 dependent protein has been shown to regulate the expression on BCL2A1, but in our studies p53 is down regulated after CAP treatment, hence no further analysis of nutlin protein were carried out. Apart from these factors BCL2A1 has been shown to be regulated by other transcription factors such as all-trans retinoic acids, WT1 or PU1. However, we found a very close correlation of the TNF expression and BCL2A1 expression in two TNBC cell lines (FIG. 5C).

TNF is a cell signaling protein (cytokine) involved in apoptotic cell death by extrinsic apoptotic signaling pathway via death domain receptors and also in the promotion and progression of cancer, including triple negative breast cancer cells. TNF mRNA expression is was significantly up-regulated after CAP treatment suggest TNF is involved in the both induction of apoptosis and in survival of breast cancer cells after CAP treatment (FIG. 1). In the case of T-47D 7 (ER+, PR+, HER2−) and SK-BR-3 (ER−, PR−, HER2+) which are the least resistance cell lines among all the cell lines treated with CAP, the expression of TNF in these cell lines are up regulated after CAP treatment but had very little detectable BCL2A1 mRNA. However, MDA-MB-231 (ER−, PR−, HER2−) and Hs574T (ER−, PR−, HER2−) TNBC cell lines which are comparatively more resistant to CAP treatment had a corelative TNF and BCL2A1 mRNA expression after treatment. Previous studies have shown upregulation of BCL2A1 protein prevents apoptosis induced by various stimuli including TNF-α, TRAIL, Fas, and chemotherapeutic agents by directly binding to tBid and BAK. Several studies have shown that increase in TNF expression induces cell survival and cancer progression. Studies have also shown that TNF-α gene knockout in TNBC cell line induces apoptosis. Furthermore, silencing of BCL2A1 by siRNA showed increased cell death within 12 hours of CAP treatment compared to CAP treatment alone group. The upregulation of BCL2A1 at lower power setting of CAP treatment increasing the survival of the cells.

Drastic reduction in G1 phase cells and an increase in S/G2 phase cells compared to control groups after CAP treatment at 80 p 5 mins is because of the histone mRNA degradation (article submitted for publication) and a pause in cell division which in turn accumulates more cells at late S phase. The phenomenon is more pronounced in cells treated with siRNA-BCL2A1 in combination with the CAP treatment suggesting that silencing BCL2A1 expression increases cell death. In the siRNA-BCL2A1-CAP treated group all most all of the cells undergo cell death by 24 hrs. at late S phase and there is a plateau phase at this stage because of the fluorescent marker proteins are oxidized and the fluorescence are retained with incomplete protein degradation. Moreover, even though there is no detectable BCL2A1 protein at the baseline level in MDA-MB-231 cells, siRNA-BCL2A1 without the CAP treatment also showed a small increase in the cells at S/G/M phase compared to siRNA control and mock groups, suggesting that there is some undetectable functioning BCL2A1.

CPI203, a BET bromodomain inhibitor that was previously shown to down regulate BCL2A1 increased cell death in when treated in combination with CAP treatment compared to treatment alone. But a pan BCL family antagonist was not so effective comparatively. CAP treatment in combination with GA did not show much potency, this could be because BCL2A1 overexpression has shown to be a drivers of intrinsic resistance to BCL-2, BCL-XL, and MCL-1 inhibitors. The only interesting exception was with BT-474 (ER+, PR+, HER2+), which show the most resistance to CAP treatment compared to all other breast cancer lines, even though BT-474 do not express BCL2A1. The resistance inducing factor in this cell line remains to be determined.

Among the induction of apoptosis gene group, ABL1 that aid in intrinsic apoptotic signaling pathway via DNA damage. ABL1 mRNA expression is down regulated throughout the 24 hrs. incubation time after CAP treatment suggesting DNA damage is enough DNA damage is not triggered after CAP treatment at 80 p 5 min for ABL1 to be up regulated. Similarly the mRNA expressions of CRADD and FADD, the genes involved in extrinsic apoptotic signaling pathway via death domain receptors is relatively unchanged or down regulated compared to baseline condition suggest that these two are not involved in the induction of apoptosis in CAP treated breast cancer cells. TP53 is a nuclear transcription factor that regulates the expression of a wide variety of genes involved in apoptosis, growth arrest, or senescence in response to genotoxic or cellular stress. AKT1 is an essential serine/threonine-specific protein kinase for multiple cellular processes essential for cellular growth, metabolism, and survival was down regulated throughout the 24 hrs. time point after CAP treated breast cancer cells. BAD induces apoptosis by inhibiting antiapoptotic BCL-2-family members—BCL-x, Bcl-2, thereby allowing two other pro-apoptotic proteins, BAK and BAX, to aggregate and induce release of cytochrome c, followed by caspase activation and apoptosis. BAD mRNA expression is relatively unchanged throughout the 24 hrs. time point suggesting that the cell death induced by CAP treatment on breast cancer cells does not involve BAD mRNA expression. BID, BIK and BNIP3 are pro-apoptotic member of the Bcl-2 superfamily and as such possess the ability to target intracellular membranes and contains the BH3 death domain. BID and BNIP3 mRNA expression were also down regulated throughout the 24 hrs time point after treatment. Whereas BIK mRNA recovery after initial down regulation suggest that BIK is involved in CAP induced cell death along with BCL2A1. BAX mRNA expression which is regulated by the tumor suppressor P53 remained at baseline levels with brief down regulation at 12 hrs time point. Both BCL2 and BAG3 mRNA were up regulated at 3 and 6 hrs incubation time point and down regulated by 12 hrs time point and again up regulated at 24 hrs incubation time but this differentially regulation in response to the CAP treatment was not statistically significant. BCL2 associated athanogene 1 programmed cell death. BCL2 like 10 (BCL2L10) gene has been shown to suppress cell apoptosis possibly through the prevention of cytochrome C release from the mitochondria, and thus preventing caspase-3 activation. Bifunctional apoptosis regulator (BFAR) a multidomain protein that was originally identified as an inhibitor of Bax-induced apoptosis. MCL1, BCL2 family apoptosis regulator (MCL1) an anti-apoptotic member of the B-cell lymphoma 2 (Bcl-2) family of apoptosis-regulating proteins, exemplifies a number of the mechanisms by which a protein's contribution to cell fate may be modified. Nucleolar protein 3 (NOL3) an anti-apoptotic protein that has been shown to down-regulate the enzyme activities of caspase 2, caspase 8 and tumor protein p53. BAG1, BCL2L10, BFAR, MCL1 and NOL3 mRNA expressions follow a similar trend by remaining at baseline levels at 3 and 6 hrs. incubation time point but is slightly down regulated at 12 hrs and up regulated at 24 hrs. incubation time point. Expect for CASP4 all the other Caspases that we analyzed were either unchanged or down regulated after CAP treatment. Caspase 4 is involved in the cascade of apoptosis execution. Caspase 4 (CASP3) protein interacts with caspase-8 and caspase-9 and is activated in the apoptotic cell both by extrinsic (death ligand) and intrinsic (mitochondrial) pathways and functions as a downstream enzyme in the caspase activation cascade. How increase in CASP4 expression either involved in cell death or survival should be determined. Among all oxidative stress genes (NOX5, GCLC, GCLM, GSR, NQO1, SQSTM1 and TXNRD1) Apolipoprotein E (APOE) showed significant up regulation after low power (80 p 5 mins) CAP treatment, however, this increase was not retained at high power CAP settings.

BCL2A1 expression plays an important role in the cell survival after CAP treatment in breast cancer cells and potentially regulated by TNF-alpha. Silencing BCL2A1 by siRNA treatment or by down regulating its expression by CPI203 treatment in combination with CAP significantly increases the potency of the CAP treatment. Based on our discovery a combination of CAP and anti-BCL2A1 treatment would be beneficial and a novel therapeutic treatment option for triple negative breast cancer and other cancers.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. A method for treating cancer comprising:
treating a patient having a cancerous tumor with a gene inhibitor pre-operatively to inhibit upregulation of a particular gene;
surgically removing the cancerous tumor;
treating the patient with the gene inhibitor CPI203 intra-operatively to inhibit upregulation of the particular gene;
applying cold atmospheric plasma to surgical margins around the area in the patient from which the tumor was surgically removed; and
treating the patient with the gene inhibitor CPI203 post-operatively.

2. A method for treating cancer according to claim 1 wherein said cancer comprises triple negative breast cancer.

3. A method for treating cancer according to claim 1 wherein said particular gene comprises BCL2A1.

4. A method for treating cancer according to claim 1 wherein said pre-operative treating a patient with the gene inhibitor CPI203 is within 24 hours of surgical removal of the cancerous tumor.

5. A method for treating cancer according to claim 1 wherein said post-operative treatment of a patient with the gene inhibitor CPI203 is within 24 hours after surgical removal of the cancerous tumor.

6. A method for treating cancer according to claim 1 wherein said applying cold plasma comprises applying cold plasma at a power setting of 15 Watts.
